(19) United States
(12) Patent Application Publication
(10) Pub. No.: US 2023/0112372 A1
(43) Pub. Date: Apr. 13, 2023

(54) PRIMATIZED RODENT

(71) Applicant: Wisconsin Alumni Research Foundation (WARF), Madison, WI (US)

(72) Inventor: Matthew Brown, Madison, WI (US)

(21) Appl. No.: 17/929,378

(22) Filed: Sep. 2, 2022

Related U.S. Application Data
(60) Provisional application No. 63/240,257, filed on Sep. 2, 2021.

(51) Int. Cl. A01K 67/027

(52) U.S. Cl. CPC A01K 67/0275; A01K 67/0271; A01K 2207/12; A01K 2217/052; A01K 2227/105; A01K 2227/108; A01K 2267/0387

(57) ABSTRACT

A primatized rodent or swine, and methods of making and using the primatized rodent or swine, are provided.

### CROSS-REFERENCE TO RELATED APPLICATIONS

This application claims the benefit of the filing date of U.S. application No. 63/240,257, filed on Sep. 2, 2021, the disclosure of which is incorporated by reference herein.

### STATEMENT OF GOVERNMENT RIGHTS

This invention was made with government support under TR002373 awarded by the National Institutes of Health. The government has certain rights in the invention.

### BACKGROUND

Hematopoietic stem cell transplantation (HSCT) is a promising therapy for malignant and non-malignant blood disorders as well as for mixed chimerism-based tolerance induction strategies in solid organ transplantation. Durable engraftment of graft hematopoietic stem and progenitor cells (HSPCs) is key in all clinical contexts of HSCT, and, to date, suboptimal engraftment/failure and concordant suboptimal immune cell reconstitution continues to be a major clinical complication limiting the utility of these therapies.

As the field of immune oncology, regenerative medicine, virology, toxicology and others continue their rapid growth, better tools are needed for researching the immune system. Because the immune systems in rodents differ from primates, screening and studying potential therapeutics in rodent models do not always translate into successful human therapeutics. To address that issue, there are commercially available mouse models where mice are genetically modified and/or irradiated to destroy their immune systems after which human immune tissue and/or stem cells are introduced to the mice to create humanized mice for studying the human immune system in rodents.

For example, researchers have reported primatized mice models for studying non-human primate immune systems in mice, analogous to the humanized mice. These primatized mice are made through engraftment of nonhuman primate hematopoietic and progenitor stem cells into mice that were irradiated to destroy their immune systems (see. e.g., Radtke et al.. 2019). However. Radtke et al. noted that this method only worked in one out of two mouse backgrounds tested.

### SUMMARY

As disclosed herein, a primatized mouse model was prepared to evaluate ontogeny-associated differences in in vivo hematopoietic stem and progenitor cell (HSPC) engraftment and/or immune cell reconstitution and to serve as a model to validate therapies prior to large-animal NHP studies. Multiple mouse host strains were intravenously injected with HSPCs from fetal and adult rhesus macaque non-human primates (NHPs) with or without co-transplantation of a NHP fetal thymus fragment in the mouse kidney capsule site. It was found that adult NHP mobilized blood HSPCs engrafted at low, but persistent, levels in immune-deficient mice harboring transgenes for human (NHP cross-reactive) GM-CSF and IL3, but not in mice with wild-type murine cytokines lacking NHP cross-reactivity. Importantly, when NHP fetal liver-derived HSPCs were injected along with thymic co-transplantation, there was significant multi-lineage immune cell engraftment, including multiple subsets of NHP B cells, T cells and myeloid cells in the mice.. These results give insights into ontogeny-associated HSPC engraftment and immune cell reconstitution differences. Additionally, incorporation of this primatized mouse model system into the pre-clinical NHP large animal research paradigm in a new opportunity for improving effective translation of HSCT and other therapies to the clinic. Other results, e.g., with cynomolgus cells, indicate that immune-deficient mice without a transgene(s) for a human cytokine(s) allow for similar results

The present disclosure thus provides for a primatized non-human vertebrate, for example a primatized rodent, e.g., mouse or rat, or other non-human primatized mammal such as a swine, which employs non-human primate embryonic tissue, e.g., non-human primate fetal hematopoietic cells such as those obtained from fetal liver, and non-human primate fetal thymus, e.g., which may serves as a site for engraftment of the hematopoietic cells, which primatized animal has a level of engraftment of, for example, greater than 25%. Non-human primates include but are not limited to apes, e.g., greater apes or lesser apes, monkeys and prosimians. In one embodiment, non-human primate fetal CD34+ cells, e.g.. without the need for further FACS sorting of CD34+ subpopulations and/or lineage depletion, are employed to prepare the primatized non-primate vertebrate. In one embodiment, the cells that are employed are CD3-depleted hematopoietic cells. In one embodiment, non-human primate fetal hematopoietic cells are obtained from fetal liver to prepare a primatized non-primate vertebrate. In one embodiment, non-human primate fetal hematopoietic cells are injected into the non-primate vertebrate, e.g.. injected into the liver of newborns. In one embodiment, the non-primate vertebrate is irradiated before transplant of a source of hematopoietic cells and of a site for engraftment and/or maturation of hematopoietic cells such as stem cells. In one embodiment, a non-primate vertebrate is not irradiated before transplant of a source of hematopoietic cells and a site for the engraftment and/or maturation of the cells. In one embodiment, the non-primate vertebrate is treated with a myeloablation agent, e.g., busulfan (chemical) rather than irradiation. In one embodiment, busulfan is administered to a NBSGW mouse prior totransplant. In one embodiment, the non-primate vertebrate is immune deficient (immune compromised), e.g., it does not need to be irradiated before transplant (implant). In one embodiment, the non-primate vertebrate is immune competent before irradiation and/or implant. In one embodiment, the mouse is immune compromised, e.g., genetically immune compromised (deficient), before irradiation and/or transplant. In one embodiment, the animal is genetically modified to allow for engraftment of hematopoietic cells in the absence of irradiation. In one embodiment, the non-primate vertebrate recombinantly expresses one or more primate, e.g., human, cytokines, e.g., IL3, GM-CSF. SCF, IL6, IL15, TPO, M-CSF, or any combination thereof, e.g., the non-primate vertebrate is a recombinant non-primate vertebrate, the genome of which has stably integrated and/or stably expressed DNA that encodes one or more primate cytokines, including non-human primate cytokines such as IL3. As used herein, an immune compromised non-primate vertebrate fails to produce one or more of T cells. B cells or NK cells without an exogenous tissue or cell source. For example, a mouse that does not produce mouse immune cells is engrafted with non-human primate tissues, resulting in a mouse that produces non-human primate immune cells. The resulting non-human primatized, non-primate vertebrate produces one or more of non-human primate T cells, B cells or NK cells. In one embodiment, the recombinant non-primate vertebrate has a genome that expresses heterologous non-human primate major histocompatibility complex (MHC) genes, e.g., a recombinant mouse that expresses human MHC, e.g., a HLA-A2 transgenic NSG mouse. In one embodiment, the non-human primatized, non-primate vertebrate expresses monkey Mamu (rhesus) or Mafa (cyno).

The present disclosure also provides for a primatized non-human vertebrate, for example a primatized rodent, e.g., mouse or rat, or other non-human primatized mammal such as a swine, which employs non-human primate embryonic tissue, e.g.. non-human primate fetal hematopoietic cells such as those obtained from fetal liver, and non-human primate fetal thymus, e.g., which may serve as a site for engraftment of the hematopoietic cells, or induced pluripotent stem cell (iPSC)-derived or embryonic stem cell (ESC)-derived hematopoietic stem or progenitor cells (HSPCs), which primatized animal has a level of engraftment of, for example, greater than 25%. Non-human primates include but are not limited to apes, e.g.. greater apes or lesser apes, monkeys and prosimians. In one embodiment, non-human primate fetal CD34+ cells, e.g., without the need for further FACS sorting of CD34+ subpopulations and/or lineage depletion, are employed to prepare the primatized non-primate vertebrate. In one embodiment, the iPSCs or ESCs are those capable of providing fetal-like HSPCs. In one embodiment, neonatal cells or tissue are obtained from a non-human primate that is up to one year in age. In one embodiment, cells or tissue are obtained from a non-human primate that is older than a neonate but not yet an adult, e.g., from 1 to 2-3 years in age. In one embodiment, adult cells or tissue are obtained from a non-human primate that is at least 2.5 years or older. In one embodiment, fetal cells or tissue are of a gestational age of at least 60 days up to about 100 days, e.g., about 80 days up to birth.

Further provided is a method of making a primatized rodent or swine, comprising: providing an immune deficient rodent or swine lacking mature T cells. B cells and/or NK cells, which rodent or swine expresses primate IL3 and/or primate GM-CSF; providing a population of cells from a fetal liver of a non-human primate which population comprises isolated CD34+ cells or which population is depleted of CD3+ cells; providing at least one portion of neo-natal of adult thymus from a non-human primate; and introducing an amount of the population of cells and the at least one portion of the thymus into the rodent or swine so as to provide a primatized rodent or swine.

The disclosure provides for a method of making a primatized non-primate vertebrate such as a rodent or swine. The method includes, in one embodiment, providing an immune deficient rodent or swine lacking mature T cells, B cells and/or NK cells, which rodent or swine expresses primate IL3 and/or primate GM-CSF; providing a population of cells from a fetal liver of a non-human primate which population comprises isolated CD34+ cells or CD3-depleted cells; and providing at least one portion of a thymus from a fetal non-human primate. In one embodiment, the hematopoietic cells, e.g., CD34+ cells or CD3-depleted cells, are expanded in vitro prior to administration, for example, expanded in the presence of one or more molecules that enhance proliferation such as SR1 or UM171. An amount of the population of cells and at least a portion of thymus are introduced (implanted) into the rodent or swine, thereby providing for a primatized rodent or swine comprising engrafted non-human primate mature T cells, B cells and/or NK cells. In one embodiment, the immune deficient rodent or swine lacks mature T cells. B cells and NK cells. In one embodiment, the rodent or swine expresses primate IL3 and primate GM-CSF. In one embodiment, the non-human primate is a monkey. In one embodiment, the monkey is a cynomologus macaque. In one embodiment, the monkey is a rhesus macaque. In one embodiment, the non-human primate is an African green monkey. In one embodiment, the non-human primate is an old world or new world non-human primate. In one embodiment, the cells are introduced via injection. In one embodiment, one or more portions of a thymus are introduced to one or more kidneys or ears of the rodent or swine. In one embodiment, the source of the cells and the thymus is the same (autologous). In one embodiment, the cells and the thymus are allogeneic to one another. In one embodiment, the cells and the thymus may also have some degree of MHC matching to one another. In one embodiment, one class I MHC and one class II MHC are matched for the thymus and CD34+ cells, which may allow for proper MHC-restriction in the chimeric T cells. In one embodiment, the cells and the thymus are xenogeneic. “Xenogenic” in this context refers to a species disparity between the thymus tissue and hematopoietic stem/progenitor cells. In one embodiment, the rodent or swine is immune deficient as a result of irradiation. In one embodiment, the rodent or swine is immune deficient as a result of one or more genetic mutations. In one embodiment, the rodent or swine expresses human IL3 and/or human GM-CSF. In one embodiment, the rodent or swine further expresses primate SCF. In one embodiment, the rodent or swine further expresses human SCF. In one embodiment, the portion of the thymus is about 0.05 mm × 3 mm, about 0.5 mm × 2 mm or about 1 mm × 1 mm. In one embodiment, two portions of thymus are introduced to the rodent or swine, each about 0.05 mm × 3 mm, about 0.5 mm × 2 mm or about 1 mm × 1 mm. In one embodiment, the population comprises about 0.05 × 10⁵ to about 9 × 10⁵ cells, about 0.1 × 10⁵ to about 7.5 × 10⁵ cells or about 0.5 × 10⁵ to about 1.5 × 10⁵ cells. In one embodiment, the population comprises 1 × 10³ up to about 5 × 10⁶ cells. e.g., about 1×10⁴ cells up to about 2×10⁶ cells. The cells may be from a fetal liver buffy coat sample or another sample that is not subjected to CD34+ purification. In one embodiment, the CD34+ cells are isolated, for example, using beads or FACS sorting. In one embodiment, the CD34+ cells are isolated using density centrifugation. In one embodiment, the rodent is a Taconic NOG-EXL mouse. In one embodiment, the rodent is a JAX NSG-SGM3 mouse. In one embodiment, the engraftment efficiency is at least about 20%. In one embodiment, the engraftment efficiency is up to about 35%. In one embodiment, the engraftment efficiency is up to about 100%. In one embodiment, the engraftment efficiency is greater than 35%. In one embodiment, the engraftment efficiency is up to about 65%. In one embodiment, the engraftment efficiency is about 35% up to about 65%.

In one embodiment, a primatized mouse may be used for xenotransplantation studies, e.g., a primitized mouse (with autologous or allogeneic or xenogeneic tissues as described herein) is transplant with xenogeneic or partially xenogeneic tissues, such as a monkey heart or heart fragment or stem cells, to assess transplant immunology response.

Further provided, in one embodiment, is a primatized rodent or swine comprising non-human primate mature T cells, B cells and/or NK cells and a portion of a thymus from a fetal non-human primate, which rodent or swine expresses primate IL3 and/or primate GM-CSF. In one embodiment, the non-human primate is a monkey. In one embodiment, the monkey is a cynomologus macaque. In one embodiment, the monkey is a rhesus macaque. In one embodiment, the thymus is implanted proximal to the kidney. In one embodiment, the cells and the thymus are autologous. In one embodiment, the cells and the thymus are allogeneic. In one embodiment, the cells and the thymus are xenogeneic. In one embodiment the cells and/or thymus are from more than one species, for example, portions of human and monkey thymus, or monkey and mouse thymus may be employed. In one embodiment, the thymus is from one monkey species and the cells are from another monkey species. In one embodiment, the thymus is from a monkey and the cells are from one or more humans. In one embodiment, the thymus is from a human and the cells are from one or more monkeys. In one embodiment, the cells or the thymus tissue, or both, are cryopreserved prior to primatized rodent or swine preparation. In one embodiment, the rodent or swine expresses human IL3 and/or human GM-CSF. In one embodiment, the rodent or swine further expresses primate SCF. In one embodiment, the rodent or swine further expresses human SCF. In one embodiment, the mouse is primatized with tissue or cells from a Mauritian cynomologus monkey, which species are an inbred population, with very limited MHC diversity. The use of MHC typed cynomologus monkey tissue can lock in place certain MHCs, which simplifies results for infectious disease or transplant therapies, allowing for large animal studies with animals of the identical MHC type

In one embodiment, a recombinant mouse that expresses human IL3 and GM-CSF, e.g., a Taconic NOG-EXL mouse, is implanted with CD34+ cells from a non-human primate fetal liver, e.g., via iv injection, and a non-human primate fetal thymus fragment, e.g., a cryopreserved thymus fragment. In contrast to other mouse models, the present primatized mouse employs a different source of immune cells, e.g., rather than using hematopoietic and progenitor stem cells isolated from bone marrow, yielding a mouse where engraftment was more stable than previously reported. Also, in contrast to other mouse models, the present primatized mouse is not transplanted with adult tissue or cells, which resulted in lower engraftment efficiencies in other mouse models. The present mouse may provide for a lower cost alternative to nonhuman primate studies, e.g., the mice could be used early in the drug screening process to improve the chances of lead compounds being effective towards primate immune systems. In one embodiment, the cells and tissue to be transplanted are from one or more monkeys, e.g., rhesus macaques.

In one embodiment, the population of CD34+ cells are obtained using magnetic beads, e.g., magnetic activated cell sorting (MACS) purification, using for instance, beads from Miltenyi Biotec, Dynabeads® from Thermofisher, or EasySep™ (Stem Cell Technologies). For example, CD34+ cells are isolated using a MACS system, with direct labeling of the CD34+ cells (e.g., with an anti-NHP CD34 antibody conjugated to APC, and optionally a secondary anti-APC antibody), or indirect staining, e.g.. with anti-CD3 to bind and remove all the T cells, leaving the remaining cells enriched for hematopoietic stem and progenitor cells.

### BRIEF DESCRIPTION OF THE FIGURES

FIG. 1. Illustration of current translational research paradigm (left), and new proposed paradigm (right) incorporating a primatized mouse model which could reduce the numbers of non-human primates (NHPs) required for biomedical research.

FIGS. 2A-2B. Engraftment of human thymus tissue in NeoThy and BLT type humanized mice. A) Human neonatal thymus is abundant (e.g., 14.75 g, shown). Membrane, adipose, and blood medium (II), then small 1 × 1 mm fragments (III) for cryopreservation. More than 1,000 small fragments suitable for transplantation can be obtained from a single thymus. B) Implanted thymus fragments develop into organoids under the kidney capsule of NBSGW hosts humanized via i.v. injection of CD34+ hematopoietic stem cells. Histological analysis of BLT (fetal) type Hu-mice (top) and NeoThy (neonatal) Hu-mice organoids (bottom) show typical anatomical features, including Hassall’s corpuscles.

FIG. 3. In vitro Functional Analysis of NHP T Cells. Mauritian cynomologus macaque peripheral blood mononuclear cells were labeled with CFSE proliferation dye and stimulated in culture for 5 days with phytohaemagglutinin (PHA) vs. unstimulated control. Stimulated cells gated on CD3+ T cells demonstrate a CFSE dye dilution by flow cytometry and classic blast-like phenotype, indicative of a functional proliferation response.

FIGS. 4A-4D. Hematopoietic and lymphopoietic potential of fetal rhesus macaque tissue. A) Tissues from a fetal 100 day gestation age rhesus macaque fetus were harvested and processed. B) Single cell suspensions were stained with anti-NHP CD34 antibody, demonstrating the presence of hematopoietic stem cells in the bone marrow and fetal liver. C) Thymus tissue was dissected into 1 mm× 1 mm fragments suitable for primatization experiments. D) Histological analysis of H+E stained thymus sections shows anatomical structures required for T cell development.

FIGS. 5A-5D. Low-Level Engraftment of Adult Mobilized Blood HSPCs in Immune-Deficient Mice. (A) Frozen mobilized blood from an adult male rhesus was processed via MACS beads to isolate CD34+ cells for injection. Post-enrichment purity in this example was 91.9%. 1 × 10⁵ - 1 × 10⁶ cells were i.v. injected per mouse for all experiments. (B) Representative flow plots of NHP mice (left) showing overall NHP-CD45 vs. Mouse-CD45 engraftment and CD20 (B cell) and CD3 (T cell) frequency of the NHP-CD45+ cells, compared to human cord blood CD34+ injected controls (right), 1 × 10⁵ CD34+ cells were injected, and 55cGY-irradiated NOG-EXL strain was used for both sets of mice. (C) NOG-EXL mice harboring human IL3 and GM-CSF transgenes were injected with 1 × 10⁶ NHP-CD34+ cells per animal, and (D) monitored for chimerism over time (representative NHP mobilized blood-injected mouse shown). Results in C and D shown at 8-11 weeks post-injection, compiled from n = 2 separate experiments. Value of ≤ 0.1 and/or < 4 positive events were considered background and listed as 0% engraftment.

FIGS. 6A-6B. Adult vs. Fetal Hematopoietic Stem and Progenitor Cell Phenotypic Differences. NHP-CD34 hematopoietic stem and progenitor cell (HSPC) purity was measured following MACS-purification of rhesus mobilized blood (left, negative control = unstained cells) and fetal liver (right, negative control = negative MACS fraction), including CD341o and CD34hi populations. Mobilized blood was from same donor but a separate purification than shown in FIG. 5.

FIG. 7. Anti-CD2 Depletion of NHP Passenger Thymocytes. Thymocytes from a fetal rhesus thymus were stained with 5 µl (XXmg) anti-CD2 antibody from loCD2 hybridoma, followed by secondary anti-rat 1 g-PE antibody. Positive staining is shown in unfilled vs. grey filled unstained control.

FIGS. 8A-8B. Adult vs. Fetal Hematopoietic Stem and Progenitor Cell Phenotypic Differences. (A) Flow cytometric analysis of adult mobilized blood, adult bone marrow, adult peripheral blood mononuclear cells (PBMCs) and fetal liver for Radtke et al., 2017 Science™, subset VII hemtopoietic stem and progenitor cell (HSPC) markers. Cells were gated on lineage (CD3-, CD20-, CD14-, CD66abce-, NKG2A-) negative, viable cells using fixable live/dead stain. (B) Non-viable cells are captured in the CD45RA+ gate (top plot), whereas viable cells are negative for CD45RA after lineage subtraction (bottom plot).

FIGS. 9A-9J. Robust Engraftment of Fetal Liver HSPCs and Thymic Tissue in NOG-EXL Mice. Two NOG-EXL mice were transplanted with fetal rhesus thymus tissue and i.v. injected with 1 × 10⁶ CD34+ cells isolated from fetal liver. (A) Overall engraftment (NHP-CD45 vs. mouse-CD45) at week 11 post-surgery is shown on the left, and the proportion of the NHP-CD45 cells expressing B cell marker CD20 and T cell marker CD3 is shown (right). (B) Engraftment kinetics are shown from week 4 post-primatization surgery to week 11. Mouse 1 was removed after week 5 due to premature death. (C) Flow cytometric analysis for adaptive and innate immune makers in the peripheral blood of NHP-mice are shown at week X, compared to adult (X year old) and fetal (X days gestation) NHP peripheral blood controls. (D) Flow cytometric analysis was performed on splenocytes. (E) NK subset analysis. (F) Basophil, neutrophil and eosinophil analyses. (G) Monocyte subsets. (H) CD20 versus CD3 analyses. (I) CD8 versus CD4 analyses. (J) Serum samples from week X NHP mice were analyzed by plex NHP Luminex panel for systemic cytokines/cytokines of interest vs. non-engrafted control mice.

FIG. 10. CCR7 Expression on NHP Mouse and Fetal NHP T Cells. Comparative flow cytometric analysis of CCR7 expression by circulating CD4⁺ T cells in the primatized NOG-EXL mouse, rhesus fetal cord blood, and adult rhesus peripheral blood.

FIGS. 11A-11E. Fetal Thymic Organoid and Tissue Resident Immune Cells. Thymic organoids formed in NHP mice, with (B) H+E staining showing clearly organized medullary and cortical regions as well as Hassall’s Corpuscles. (C) The organoid was stained by immunohistochemistry for NHP-CD3 to determine the presence of developing T cells, and by flow cytometry (D) to evaluate single and double positive cells (NHP-CD4 and/or CD8). (E) Paired H&E and anti-CD3 IHC demonstrating non-human primate CD3+ T cells within the capillaries and/or lacteal of intestinal villi. Inset enlarged to show detail.

FIG. 12. NHP Thymocytes Isolated from Fetal Thymus.

FIGS. 13A-13B. Hematopoietic Engraftment in NHP Mouse Bone Marrow and Secondary Transplantation. (A) Bone marrow was harvested from NHP mouse femurs and analyzed for hematopoietic stem and progenitor markers. (B) Bone marrow was harvested from NHP mouse femurs and transplanted into secondary, naïve NSG-SGM3 mice, which have human IL3 and GM-CSF transgenes, similar to the NOG-EXL, but also harbor a human SCF transgene. 2 × 10⁶ total cell injected per animal, n = 8, 5 males and 3 females. Values given as a percentage of total engrafted NHPCD45+ cells.

FIG. 14. Secondary Hematopoietic Engraftment in NOG-EXL Mice. Isolated NHP mouse bone marrow samples were transplanted into secondary, naive NOG-EXL mice (2 × 10⁶ total cells per animal) and monitored for secondary engraftment. N = 4 male mice, two mice died between week 4 and 5.

FIGS. 15A-15C. NHP Mice Immune Cell Function. (A) Splenocytes from NHP mice were harvested, labeled with Cell Trace Violet (CTV) and then stimulated with 1× PMA/lonomycin or PHA (10 µg/ml). (B) A mixed lymphocyte reaction (MLR) was performed with isolated NHP mouse splenocytes mixed in a 2:1 dilution with counter-labeled, irradiated adult PBMC; IL-2 (100 ng) was added for one condition as an additional inflammatory stimulus. (C) Cells were assayed by intracellular flow cytometry for interferon gamma (IFN-γ) and tumor necrosis factor alpha (TNF-α) production.

FIGS. 16A-16D. Low-Level Engraftment of Adult Mobilized Blood HSPCs in Immune-Deficient Mice. (A) Frozen mobilized blood from a male adult rhesus was processed via MACS beads to isolate CD34⁺ cells for injection. Post-enrichment purity in this example was 91.9%. 1×10⁵-1×10⁶ cells were IV injected per mouse for all experiments. (B) Representative flow plots of NHP mice (left) showing overall NHP-CD45 vs Mouse-CD45 engraftment and CD20⁺ (B cell) and CD3⁺ (T cell) frequency of the NHP-CD45⁺ cells, compared to human cord blood CD34⁺ injected controls (right), 1×10⁵CD34⁺ cells were injected, and 55cGY-irradiated NOG-EXL strain was used for both sets of mice. (C) NOG-EXL mice harboring human IL3 and GM-CSF transgenes were injected with 1×10⁶NHP-CD34⁺ cells per animal, and (D) monitored for chimerism over time (representative NHP mobilized blood-injected mouse shown). Results in C and D shown at 8-11 weeks post-injection, compiled from n=2 separate experiments. Value of ≤0.1 and/or <4 positive events were considered background and listed as 0% engraftment.

FIG. 17. Adult vs Fetal Hematopoietic Stem and Progenitor Cell Phenotypic Differences. NHP-CD34 hematopoietic stem and progenitor cell (HSPC) purity was measured following MACS-purification of rhesus mobilized blood (left, negative control = unstained cells) and fetal liver (right, negative control = negative MACS fraction), including CD34lo and CD34hi populations. Mobilized blood was from same donor but a separate purification than shown in FIG. 16.

FIGS. 18A-18G. Robust Engraftment of Fetal Liver HSPCs and Thymic Tissue in NOG-EXL Mice. Two NOG-EXL mice were transplanted with fetal rhesus thymus tissue and IV injected with 1×10⁶ CD34⁺ cells isolated from fetal liver. (A) Overall engraftment (NHP-CD45 vs mouse-CD45) at week 11 post-surgery is shown on the top, and the proportion of the NHP-CD45 cells expressing B cell marker CD20 and T cell marker CD3 is shown on the bottom. (B) Engraftment kinetics are shown from week 4 post-primatization surgery to week 11. Mouse 1 was removed after week 5 due to premature death. (C) Flow cytometric analysis for adaptive and innate immune makers in the peripheral blood of NHP-mice are shown at week 20, compared to adult (15 year old) and fetal (96 days gestation) NHP peripheral blood controls. (D) B cell populations were assess, as well as (E) NK cells, (F) granulocyte subsets, (G) and monocyte subsets. Flow cytometric analysis was performed on cervical, axillary, inguinal, and mesenteric lymph nodes and splenocytes for overall NHP chimerism and analysis of T cell CD4⁺ and CD8⁺ subsets. (E) Serum samples from NHP mice 12-16 weeks post-primatization were analyzed by 29-plex NHP Luminex panel for precense of cytokines and chemokines vs control that received a thymus implantation surgery but no CD34⁺ injection.

FIGS. 19A-19E. Fetal Thymic Organoid and Tissue Resident Immune Cells. Thymic organoids formed in engrafted NHP mice (A) shown grossly, with (B) H+E staining including well-organized medullary and cortical regions, as well as Hassall’s corpuscles, consistent with native thymic histology. (C) Anti-CD3 (rhesus) staining identifies the presence of diffuse T cell distribution within the cortex and medulla, which is supported by (D) flow cytometric analysis of CD4⁺ and CD8⁺, including developing double positive CD4⁺CD8⁺ subsets within the thymic organoid of a fully engrafted primatized mouse. (E) Cross-sectional H+E and anti-CD3 stained histology reveals tissue resident T and non-T lymphocytes within the intestinal villi of the mature NHP mouse.

FIGS. 20A-20B. Hematopoietic Engraftment in NHP Mouse Bone Marrow and Secondary Transplantation. (A) Bone marrow was harvested from NHP mouse femurs and analyzed for hematpoietic stem and progenitor markers (lineage negative CD38(lo)CD34+ and CD38(lo)CD34+CD45RA-CD90+ HSPC subsets, values given as percentage of NHP-CD45+ cells). (B) Total bone marrow cells were harvested from NHP mouse femurs and transplanted via IV injection into secondary, naive NSG-SGM3 mice, which have human IL3 and GM-CSF transgenes, similar to the NOG-EXL. but also harbor a human SCF transgene. 2×10⁶ total cells injected per animal, n=8, 5 males and 3 females.

FIGS. 21A-21B. NHP Mice Immune Cell Function. Splenocytes from NHP mice were harvested, labeled with Cell Trace Violet (CTV) and then stimulated with 1x PMA/Ionomycin or PHA (10 ug/ml), which (A) resulted in robust T cell proliferation [CTV(lo)] in culture. A mixed lymphocyte reaction (MLR) was performed with isolated NHP mouse splenocytes mixed in a 2:1 dilution with counter-labeled, irradiated adult NHP PBMC (B) yielding multiple cycles of in vitro T cell proliferation in response to allogenic stimulation; IL-2 (100 ng) was added for one condition as an additional inflammatory stimulus present in the post-transplantation microenvironment. At the termination of the MLR, cells were additionally assayed by intracellular flow cytometry for interferon gamma (IFN-γ) and tumor necrosis factor alpha (TNF-α) production.

FIGS. 22A-22D. Hematopoietic and Lymphopoietic Potential of Fetal Rhesus Macaque Tissue. (A) Tissues from a fetal 100 day gestation age rhesus macaque fetus were harvested and processed. (B) Single cell suspensions were stained with anti-NHP CD34 antibody to identify the presence of hematopoietic stem/progenitor cells in the bone marrow and fetal liver. (C) Thymus tissue was dissected into 1 mm × 1 mm fragments suitable for primatization experiments. (D) Histological analysis of H+E stained thymus sections shows anatomical structures required for T cell development (medulla, cortex, Hassall’s corpuscles).

FIG. 23. Anti-CD2 Depletion of NHP Passenger Thymocytes. Thymocytes from a fetal rhesus thymus were stained with 36 ug anti-CD2 antibody from loCD2 hybridoma, followed by secondary anti-rat Ig-PE antibody. Postive staining is shown in unfilled vs grey filled unstained control.

FIGS. 24A-24B. Adult vs Fetal Hematopoietic Stem and Progenitor Cell Phenotypic Differences. (A) Flow cytometric analysis of adult mobilized blood, adult bone marrow, adult peripheral blood mononuclear cells (PBMCs) and fetal liver. Top row were lineage negative (CD3⁻, CD20⁻, CD14⁻, CD66abce⁻, NKG2A⁻), and viable cells using fixable live/dead stain. Group1 gate is gated base on CD117 and CD45RA staining (2^{nd} row). Next two rows are cells in CD117⁺ CD45RA⁻ and CD117⁻ CD45RA⁻ quadrants. (B) CD45RA⁺ events in Group I (top plot) were removed by the viability gate (bottom plot).

FIGS. 25A-D. Flow cytometric analyses of NK subsets (A). CD20 versus CD3 expression (B), CD8 versus CD4 expression (C) and CCR7 expression on NHP mouse and fetal NHP T Cells (D).

FIG. 26. NHP Thymocytes Isolated from Fetal Thymus. Flow cytometric analysis of CD4⁺, CD8⁺, and double positive CD4⁺CD8⁺ subsets within the native thymus of a fetal rhesus macaque.

FIG. 27. Secondary Hematopoietic Engraftment in NOG-EXL Mice. Isolated NHP mouse bone marrow samples were transplanted via IV injection into secondary, naive NOG-EXL mice (2×10⁶ total cells per animal) and monitored for engraftment over the course of 7 weeks. N=4 male mice, two mice died between week 4 and 5.

FIG. 28. Overview of Exemplary Comparisons and Screens in Primatized Mouse.

FIGS. 29A-29B. A) Thymic organoid from mouse 1191LLRR, harvested at 11 weeks post-primatization (NOG-EXL strain). B) Cynomologus macaques can be used to create NHP mice and primatized mice were sacrificed at 11-16 weeks post-primatization. On left, cynomologus macaque thymic organoid from mouse 1250L, harvested 11 weeks post-primatization (NBSGW strain) and on right, cynomologus macaque thymic organoid from mouse 1191LLR, harvested 16 weeks post-primatization (NOG-EXL strain).

### DETAILED DESCRIPTION

The creation of humanized mouse models by grafting human hematopoietic stem and progenitor cells (HSPCs) into immune-deficient mouse hosts has uncovered important lessons about human hematopoietic engraftment and leukocyte development. In recent years, humanized mice have become a useful tool for pre-clinical experimental study of human immune function. In a similar way, non-human primates (NHPs), such as the rhesus (Macaca mulatta) and cynomologus (Macaca fascicularis) macaques serve as important large-animal models for pre-clinical translational studies involving novel therapeutic or diagnostic interventions given their biologic similarities to humans. However, the high cost and prolonged timeline of acquisition, husbandry, and experimentation in NHPs is a critical barrier to their effective and efficient use in large-scale biomedical research. A primatized model would allow for investigation of the factors that influence NHP hematopoietic engraftment, enabling more efficient large-animal studies for transplantation immunology and virology research, as well as the study of ontogeny-associated hematopoietic engraftment, immunologic development, and leukocyte function.

There have been descriptions of NHP-derived hematopoiesis and leukocyte development in immune-deficient mice (Binhazim et al., 1996; Radtke et al., 2019). However, prior to the present disclosure, there have been no reports of BLT/NeoThy-type primatization utilizing intravenous (IV) injection of HSPCs with surgical transplantation of thymus tissue. Binhazim et al. (1996) transplanted fetal hematolymphoid tissue into first-generation immune deficient SCID mice, which yielded low level chimerism but lacked development of non-T leukocytes, likely due to the suboptimal nature of the mouse strain and/or the lack of direct injection of a purified NHP HSPC source. Radtke et al. (2019) utilized a powerful next-generation host, the MISTRG mouse, harboring multiple human hematopoietic transgenes, including M-CSF, GM-CSF. and IL-3. Despite their seminal success in inducing robust hematopoietic engraftment, durable establishment of chimerism relied on injection of a rare FACS-sorted HSPC population, resulting in a low-throughput model, complicating its feasibility for large-scale studies. Furthermore, although these primatized mice did develop T lymphocytes, they harbored limited functional capacity due to an absence of NHP thymic epithelium during maturation, resulting in a suboptimal model for translational characterization of MHC-restricted immune responses in NHPs.

Humanized mice are made through transplantation of fetal or neonatal human thymus tissue along with injection of hematopoietic and progenitor cells. For example, Brown et al. (2018) disclose that humanized mice prepared using neonatal tissue were more stable than humanized mice prepared using fetal tissue. Surprising, as disclosed herein, non-human primatized mice prepared using fetal tissue were more stable (durable) than non-human primatized mice prepared using neonatal tissue ). Moreover, non-human primatized mice as disclosed herein, prepared using fetal tissue, showed enhanced T cell engraftment, e.g.. as shown by CD4/CD8 ratios that are within a normal range and/or by comparing the number of cells that are injected versus the frequency of CD45 cells derived from the implanted cells, which was unexpected.

For example, at 10 weeks post-engraftment, the disclosed primatized mouse saw approximately 80-90% NHP-CD45 engraftment versus the 20-30% engraftment in Radtke et al. (2019.) Therefore, the present method results in approximately 3-4 fold increase in engraftment compared to the mouse in Radtke et al. Moreover, engraftment of CD45RAnegCD90+ cells in the disclosed mouse was 16.6% versus 0.45% engraftment of CD45RAnegCD90+ cells in the Radtke et al. mouse.

Horn et al. (2003) showed only 5% NHP-CD45 engraftment and it did not last past 12 weeks. And while Horn et al. did secondary engraftment studies, only ∼2% chimerism was observed in secondary animals versus about ~50% chimerism in the secondary engraftment studies disclosed herein at 7 weeks. The low values in Horn et al. indicate that they had few if any long-term HSCs engraft in their animals, whereas the higher values disclosed herein indicate significant engraftment of the desired cells.

Gori et al. (2012) prepared cynomogolous HSPCs via iPS cells but do not disclose mouse transplant experiments. Abdel et al (2015) used those cells and transplanted them into NSG mice. The cynomologus iPSC-HSPCs engrafted in NSG mice at very low levels (less than 1%).

As noted above, non-human primates (NHPs) are a powerful translational research model. However, for new investigators seeking to utilize NHPs in their research, the high cost of purchasing, housing, and caring for primates is a critical barrier. This financial burden also impedes acquisition and replication of statistically significant results. As an alternative model, investigators in the fields of transplant immunology and virology are increasingly using humanized mice (Hu-mice) for preclinical studies. Hu-mice, created by engrafting human immune tissues into immune-deficient mouse hosts, can model patient T cell responses to therapeutic interventions and are advantageous due to their tractability and relative low cost vs NHPs. While Hu-mice quite accurately model human T cell biology, other components of their immune system (e.g., B and NK cells) can be phenotypically and functionally suboptimal. Therefore, in order to robustly evaluate therapies in the context of an entire functional immune system relevant to human biology, there is still a need for full-scale NHP studies prior to initiating clinical trials. The present model may be useful to analyze, for example, NHP-specific T cell-mediated immunity as the model results in long-term engraftment of primate immune cells. Such durable chimerism can be utilized in studies of therapeutic interventions where T cells play a central mechanistic role. This approach would allow for larger NHP cohort sizes and lower overall costs/number of animals than if all the therapies were tested in NHPs without the informative primatized mouse preliminary studies. Additionally, a primatized mouse model enables investigations into age-related changes in T cell phenotype and function.

### Exemplary Embodiments

In one embodiment, the disclosure provides for a method of making a primatized rodent, such as a mouse, rat, squirrel, hamster, porcupine or beaver. In one embodiment, the method includes providing an immune deficient mouse lacking mature T cells. B cells and/or NK cells, which mouse optionally expresses primate IL3 and/or primate GM-CSF; providing a population of cells from a fetal liver of a non-human primate which population comprises isolated CD34+ cells; and providing at least one portion of a thymus from a fetal non-human primate. An amount of the population of cells and at least a portion of thymus are introduced (implanted) into the mouse, thereby providing for a primatized mouse comprising engrafted non-human primate mature T cells, B cells and/or NK cells. In one embodiment, the immune deficient mouse lacks mature T cells, B cells and NK cells. In one embodiment, the mouse expresses primate IL3 and primate GM-CSF. In one embodiment, the non-human primate is a monkey. In one embodiment, the monkey is a cynomologus macaque. In one embodiment, the monkey is a rhesus macaque. In one embodiment, the cells are introduced via injection. In one embodiment, one or more portions of a thymus are introduced to one or more kidneys or ears of the mouse. In one embodiment, the source of the cells and the thymus is the same (autologous). In one embodiment, the cells and the thymus are allogeneic. In one embodiment, the cells and the thymus are xenogeneic. In one embodiment, the mouse is immune deficient as a result of irradiation. In one embodiment, the mouse is immune deficient as a result of one or more genetic mutations. In one embodiment, the mouse expresses human IL3 and/or human GM-CSF. In one embodiment, the mouse further expresses primate SCF. In one embodiment, the mouse further expresses human SCF. In one embodiment, the portion of the thymus is about 0.05 mm × 3 mm, about 0.5 mm × 2 mm or about 1 mm × 1 mm. In one embodiment, two portions of thymus are introduced to the mouse, each about 0.05 mm × 3 mm, about 0.5 mm × 2 mm or about 1 mm × 1 mm. In one embodiment, the population comprises about 0.05 × 10⁵ to about 9 × 10⁵ cells, about 0.1 × 10⁵ to about 7.5 × 10⁵ cells or about 0.5 × 10⁵ to about 1.5 × 10⁵ cells. In one embodiment, the CD34+ cells are isolated using beads. In one embodiment, the CD34+ cells are isolated using density centrifugation. In one embodiment, the mouse is a Taconic NOG-EXL mouse. In one embodiment, the mouse is a NSG-SGM3. In one embodiment, the engraftment efficiency is at least about 20%. In one embodiment, the engraftment efficiency is up to about 35%.

Further provided is a primatized mouse comprising non-human primate mature T cells, B cells and/or NK cells and a portion of a thymus from a fetal non-human primate, which mouse expresses primate IL3 and/or primate GM-CSF. In one embodiment, the non-human primate is a monkey. In one embodiment, the monkey is a cynomologus macaque. In one embodiment, the monkey is a rhesus macaque. In one embodiment, the thymus is proximal to the kidney. In one embodiment, the cells and the thymus are autologous. In one embodiment, the cells and the thymus are allogeneic. In one embodiment, the cells and the thymus are xenogeneic. In one embodiment, the mouse expresses human IL3 and/or human GM-CSF. In one embodiment, the mouse further expresses primate SCF. In one embodiment, the mouse further expresses human SCF.

### Exemplary Method of Making a Primatized Mouse

To prepare a robust non-human primate immune system in an immune deficient mouse host (e.g., the Taconic NOGEXL strain), CD34+ hematopoietic stem/progenitor cells obtained from a rhesus macaque are injected ono the mouse, and a surgery is performed to implant a rhesus macaque thymus fragment) into the immune-deficient mouse which expresses one or more human cytokines, e.g., the mouse is a transgenic mouse expressing the one or more human cytokines. The mouse may reduce the number of primates used in biomedical research, and make primate research more impactful and less costly by improving the statistical significance of findings (e.g., by testing therapies first in primatized mice, then using the best candidates for large animal studies with higher n value and fewer total animals). The thymic fragment surgery may aid in the engraftment of T cells in the animals. The present model is likely to be more widely useful than the existing models and allows researchers to conduct non-human primate studies at entities that do not have Primate Research Centers and/or if they do not have a budget large enough to purchase, house, and experiment on large-animal primates.

### Exemplary Embodiments

In one embodiment, a method of making a primatized rodent or swine is provided. The method includes providing an immune deficient rodent or swine lacking mature T cells. B cells and/or NK cells, which rodent or swine optionally expresses primate IL3 and/or primate GM-CSF; providing a population of cells from a fetal liver of a non-human primate which population comprises isolated CD34+ cells or which population is depleted of CD3+ cells, or a population which comprises hematopoietic stem or progenitor cells obtained from induced pluripotent stem cells or embryonic stem cells; providing at least one portion of a thymus from a fetal, neonatal or adult non-human primate; and introducing an amount of the population of cells and the at least one portion of the thymus into the rodent or swine so as to provide a primatized rodent or swine. In one embodiment, the primatized rodent or swine comprises mature T cells, B cells and/or NK cells. In one embodiment, the immune deficient rodent or swine lacks mature T cells, B cells and NK cells. In one embodiment, the rodent or swine expresses primate IL3 and primate GM-CSF. In one embodiment, the non-human primate is a monkey. In one embodiment, the monkey is a cynomolgus macaque or a rhesus macaque. In one embodiment, the cells are introduced via injection. In one embodiment, the thymus is introduced to a kidney or an ear of the rodent or swine. In one embodiment, the source of the cells and the thymus is the same. In one embodiment, the cells and the thymus are allogeneic. In one embodiment, the cells and the thymus are xenogeneic. In one embodiment, the rodent or swine is immune deficient as a result of irradiation. In one embodiment, the rodent or swine is immune deficient as a result of one or more genetic mutations. In one embodiment, the rodent or swine expresses human IL3 and/or human GM-CSF. In one embodiment, the rodent or swine further expresses primate SCF. In one embodiment, the rodent or swine further expresses human SCF. In one embodiment, the at least one portion of the thymus is about 0.05 mm × 3 mm, about 0.5 mm × 2 mm or about 1 mm × 1 mm. In one embodiment, at least two portions or up to five portions of thymus are introduced. In one embodiment, the population comprises about 0.05 × 10⁵ to about 9 × 10⁵ cells, about 0.1 × 10⁵ to about 7.5 × 10⁵ cells or about 0.5 × 10⁵ to about 1.5 × 10⁵ cells. In one embodiment, the CD34+ cells are enriched using beads. In one embodiment, the CD34+ cells are isolated using density centrifugation. In one embodiment, the rodent is a NCG. NSG or NOG mouse. In one embodiment, the rodent is a NSG-SGM3, NOG-EXL or NBSGW mouse. In one embodiment, the rodent is a NSG-IL15 or NOG-IL6 mouse. In one embodiment, the engraftment efficiency is at least about 20%. In one embodiment, the engraftment efficiency is up to about 35% to 65%. In one embodiment, the thymus is from a fetus and the population comprises isolated CD34+ cells or is depleted of CD3+ cells. In one embodiment, the population comprises the hematopoietic stem or progenitor cells.

In one embodiment, a primatized rodent or swine is provided comprising non-human primate T cells, B cells and/or NK cells and a portion of a thymus from a fetal non-human primate, which rodent or swine expresses primate IL3 and/or primate GM-CSF. In one embodiment, the rodent or swine comprises mature T cells, B cells and/or NK cells. In one embodiment, the non-human primate is a monkey. In one embodiment, the monkey is a cynomologus macaque or a rhesus macaque. In one embodiment, the thymus is proximal to the kidney. In one embodiment, the cells and the thymus are autologous. In one embodiment, the cells and the thymus are allogeneic to each other, e.g., from different monkeys of the same species. In one embodiment, the cells and the thymus are xenogeneic to each other, e.g., from monkeys of two different species, or from a human and a monkey, e.g., cynomolgus. In one embodiment, the rodent or swine expresses human IL3 and/or human GM-CSF. In one embodiment, the rodent or swine further expresses primate SCF. In one embodiment, the rodent or swine further expresses human SCF.

The invention will be further described by the following non-limiting examples.

### EXAMPLE 1

A primatized mouse model allows for rigorous and efficient evaluation of non-human primate (NHP) T cell-mediated immune responses to therapeutic interventions. Conducting preliminary in vivo NHP research studies in a small-scale and relatively inexpensive primatized mouse model, prior to initiation of costly large-scale NHP experiments, would be beneficial for translational research in the areas of transplantation immunology, virology, pluripotent stem cell biology, and hematopoiesis. Testing hypotheses first on this smaller-scale model could help ensure that only the most promising therapies will move forward into traditional rhesus macaque or other NHP in vivo studies. Data from primatized mice are beneficial in that that leads to NHP studies that are more rigorous and reproducible than existing studies, and that opportunities for generating statistically significant data will increase. For example, when planning traditional NHP studies, researchers having data from a primatized mouse could justify using fewer experimental groups with larger cohort sizes powered for statistical significance if the design was informed by in vivo NHP-centric preliminary data from primatized mice rather than from mouse models and/or in vitro data alone. Thus, a primatized mouse model could significantly decrease the financial costs associated with NHP research, and make NHP experiments more effective for validating promising new treatments for human patients. Additionally, a primatized mouse model could be used in studies in T cell development that are not possible to achieve with existing models.

Immune-compromised mice are implanted with primate hematopoietic and lymphopoietic tissues and the chimeric primate cells that engraft in the mice are characterized. For example, NHP T cell phenotype and function is determined from cells obtained from fetal, neonatal and adult animals and immune-compromised mice are primatized, with NHP hematopoietic and lymphopoietic tissues via i.v. injection of NHP bone marrow-derived hematopoietic stem cells, combined with surgical transplantation of thymus tissue. The resulting non-human primatized animals have prolonged engraftment of immune cells, including functional T cells

NHP experimentation is well established in biomedical research, owing to the high degree of genetic homology between NHPs and humans, and to the concordant utility of NHPs for modeling the complex in vivo environment of a typical patient. Nonetheless, these powerful models are not widely available due to regulatory restrictions and, importantly, due to the great financial costs involved in purchasing, housing, and caring for NHPs during extended studies. These costs are a barrier for new investigators seeking to utilize NHPs in their research, and impede acquisition and replication of statistically significant results (Evans & Silvestri, 2013). As an alternative model, investigators in the fields of transplant immunology and virology are increasingly using humanized mice (Hu-mice) preclinical studies. Hu-mice are created by engrafting human immune cells into an immune-deficient mouse host. While Hu-mice are a high-fidelity model of human T cell biology, other components of their immune system (e.g. B and NK cells) can be phenotypically and functionally suboptimal (Rongvaux et al., 2014; Herndler-Brandstetter et al., 2017; Brown et al., 2018). Therefore, in order to robustly evaluate therapies in the context of an entire functional immune system relevant to human biology, in many cases there is still a need for costly full-scale NHP studies prior to initiation of clinical trials.

A durably engrafted, primatized mouse model, e.g., a BLT/NeoThy type primatized mouse model, with a thymus tissue transplant, is provided. The phenotype and effector function of T cells from the primatized mice are characterized at multiple time points, and reproducible differences are discerned vs ex vivo NHP blood. The data include changes in T cell repertoires in effector phenotype and function over the course of animal development.

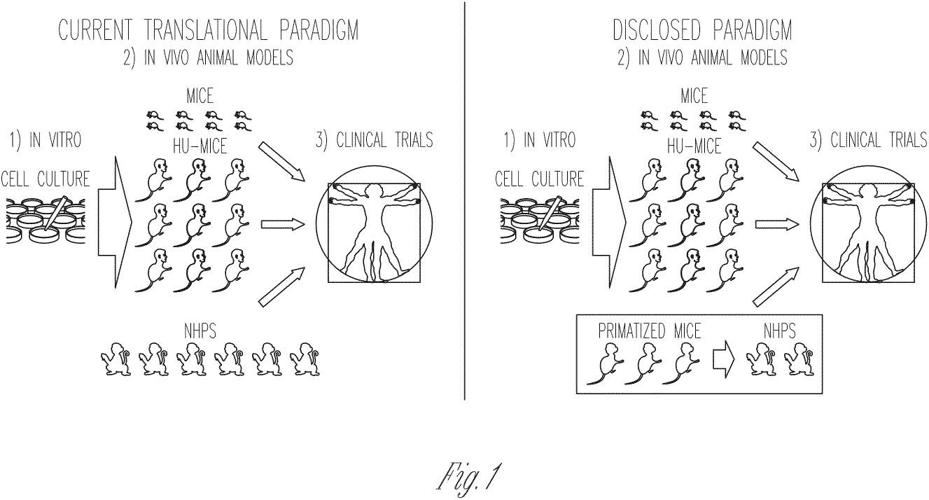

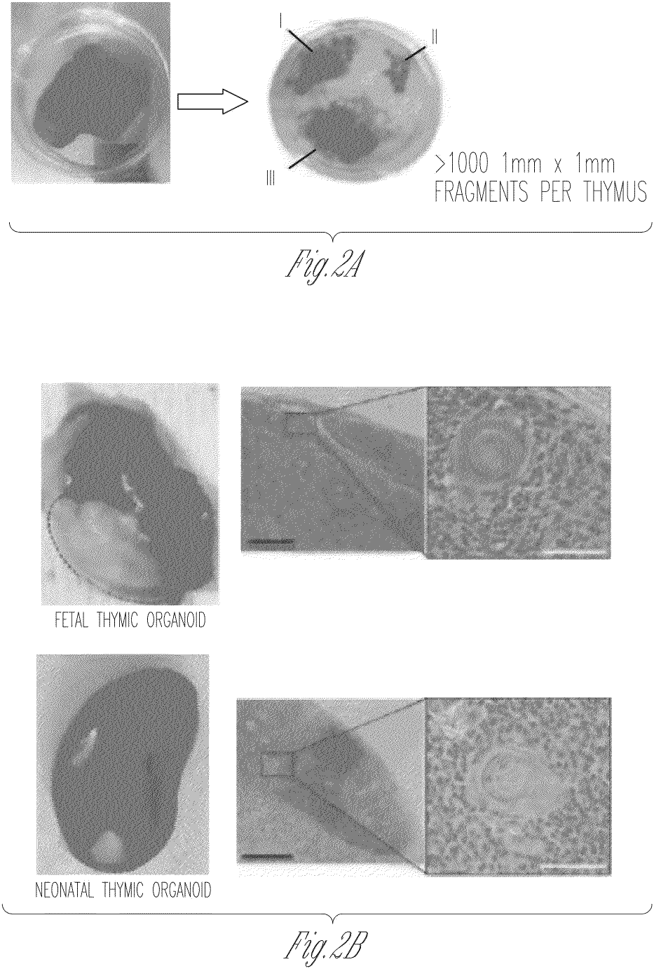

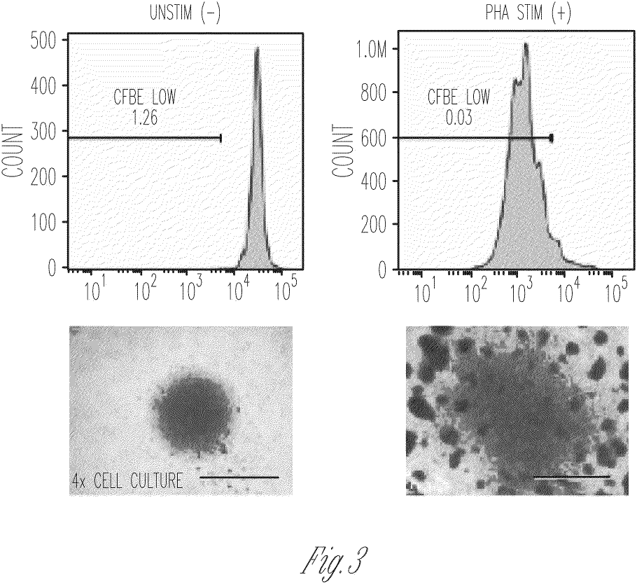

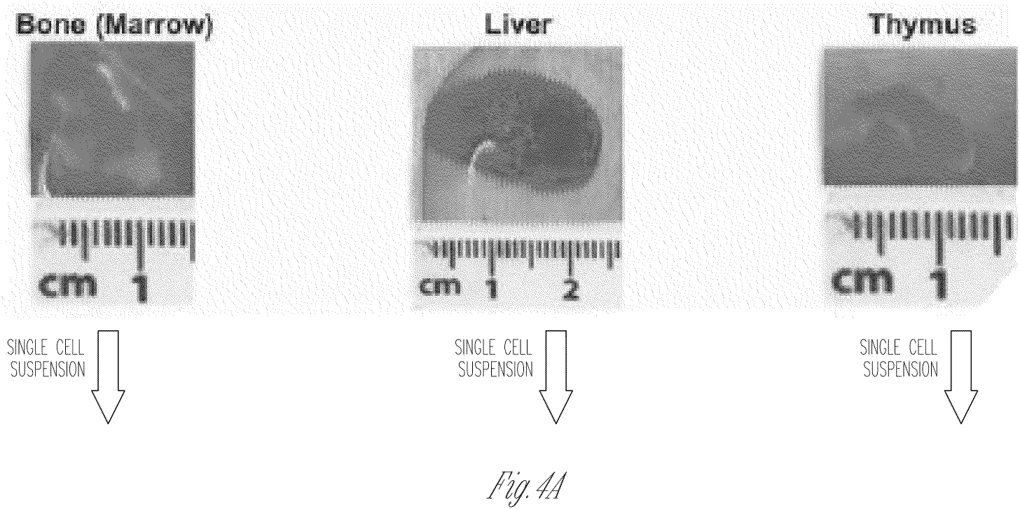

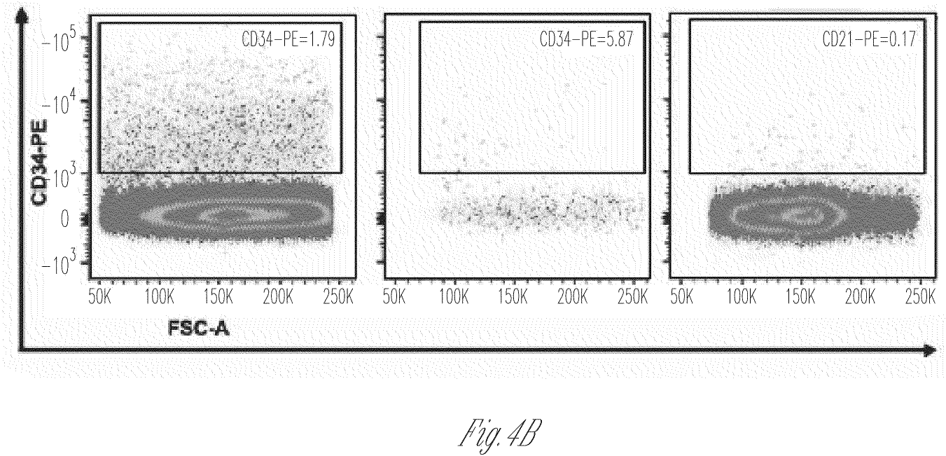

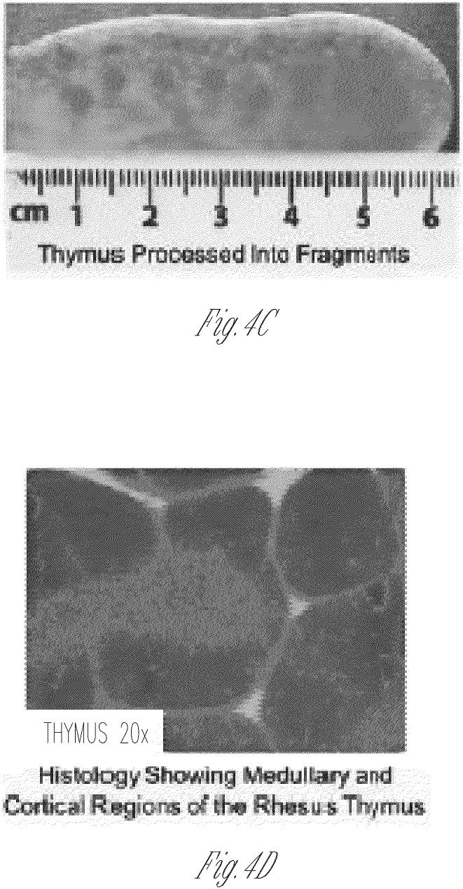

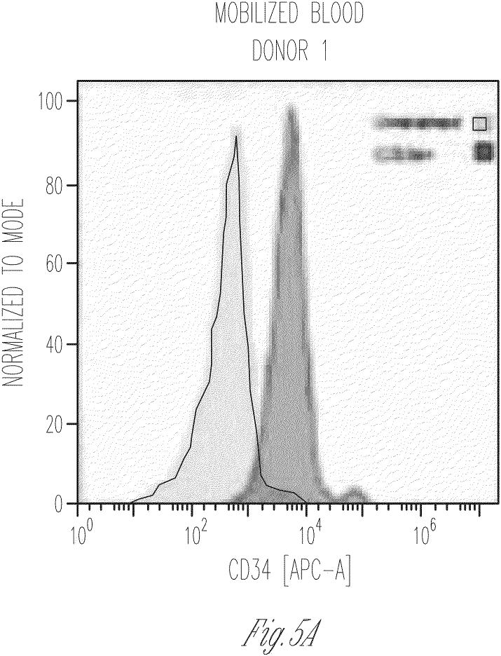

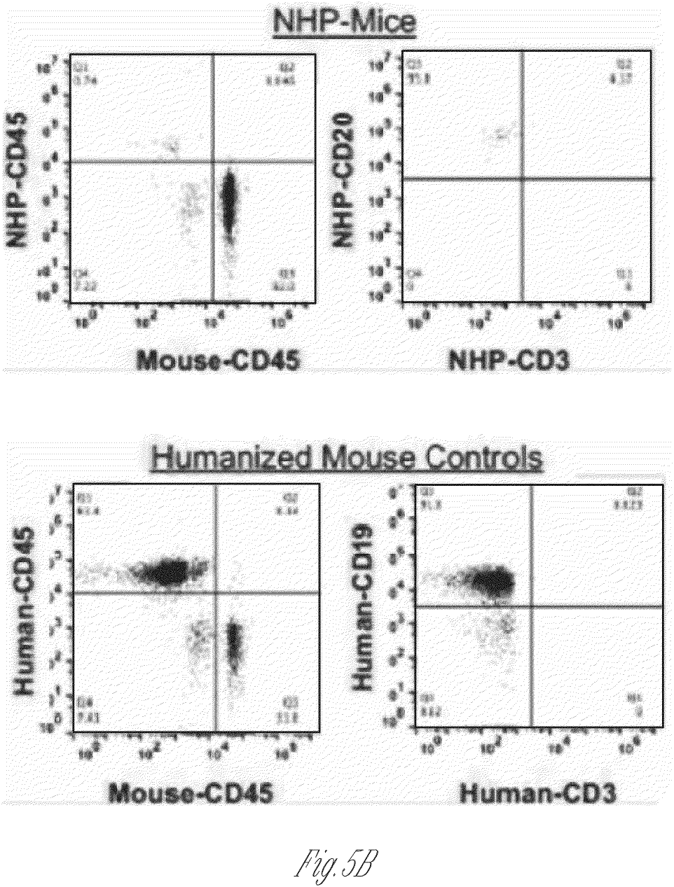

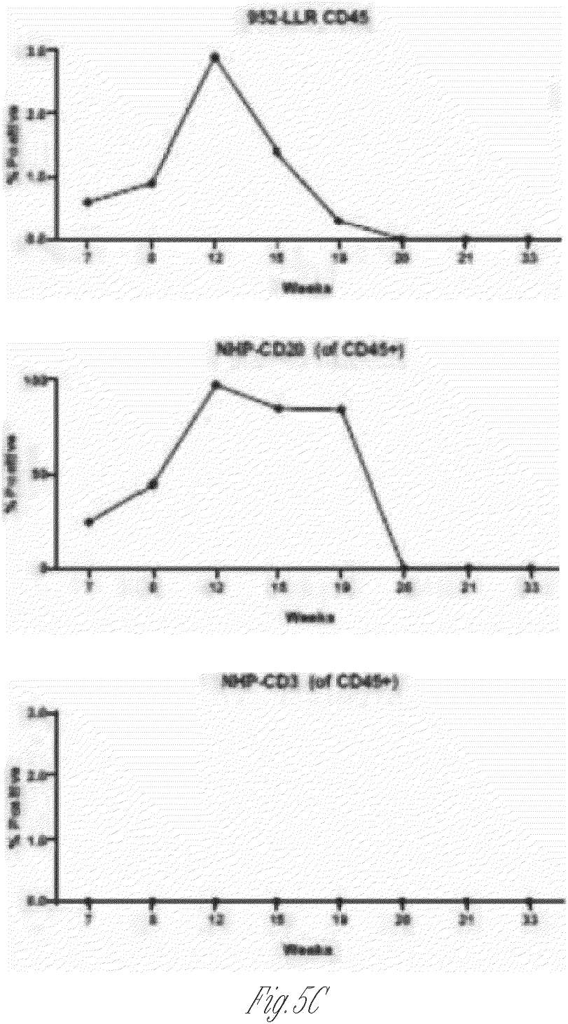

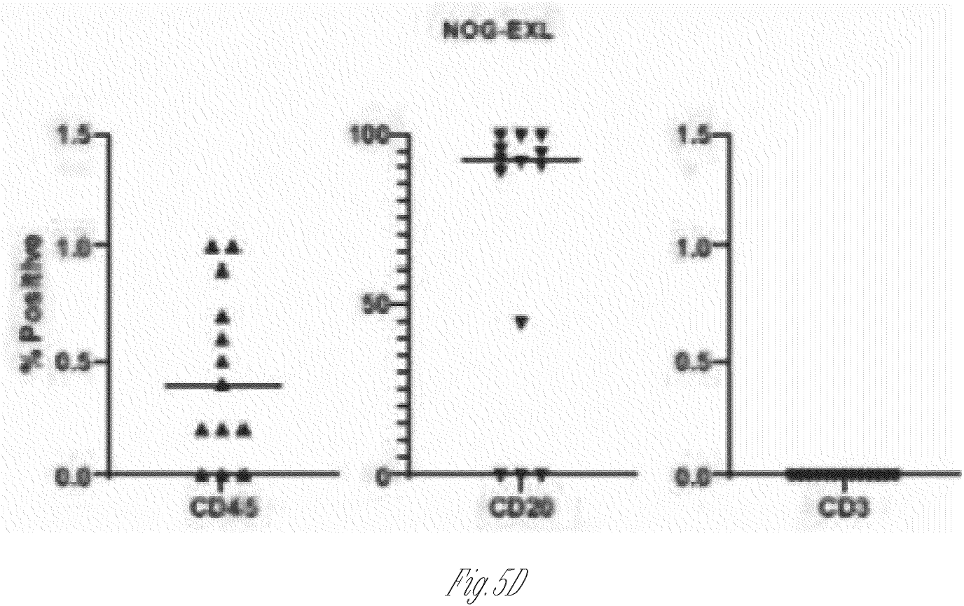

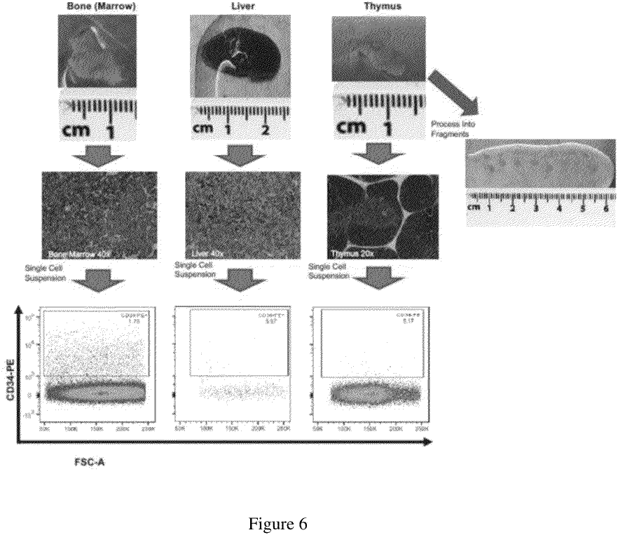

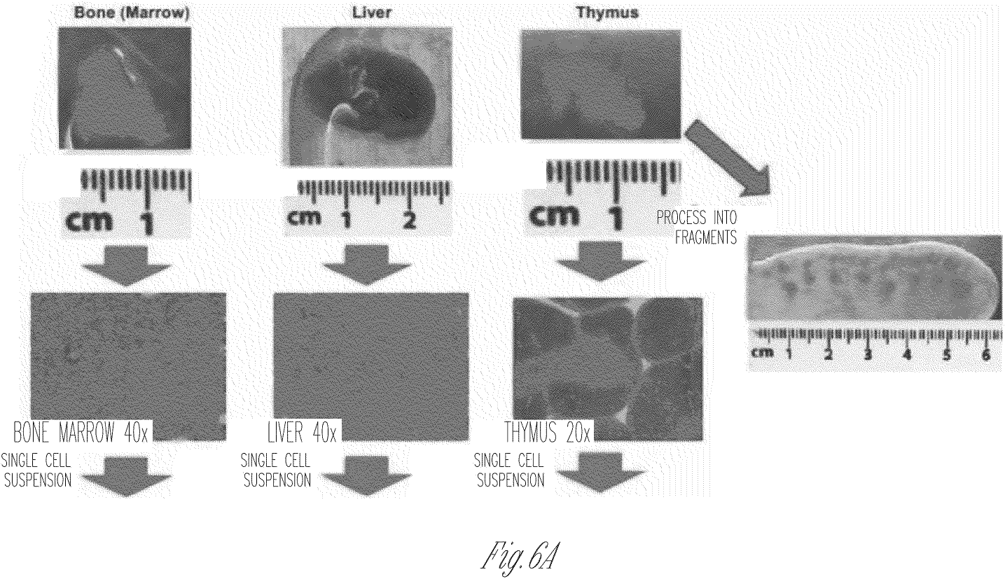

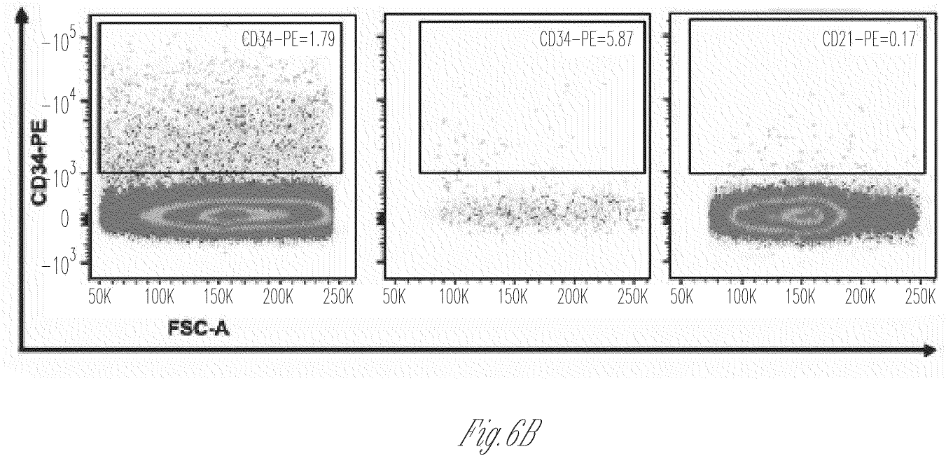

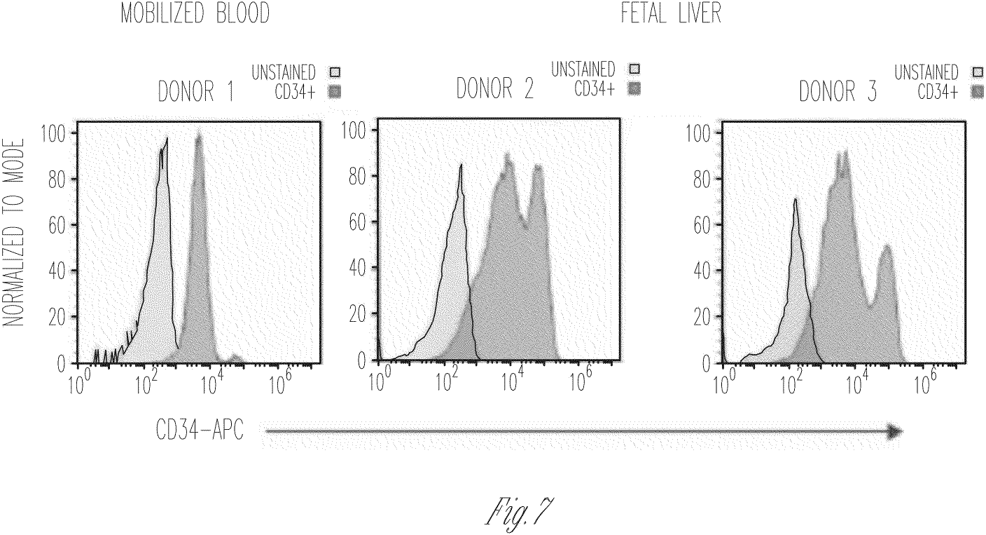

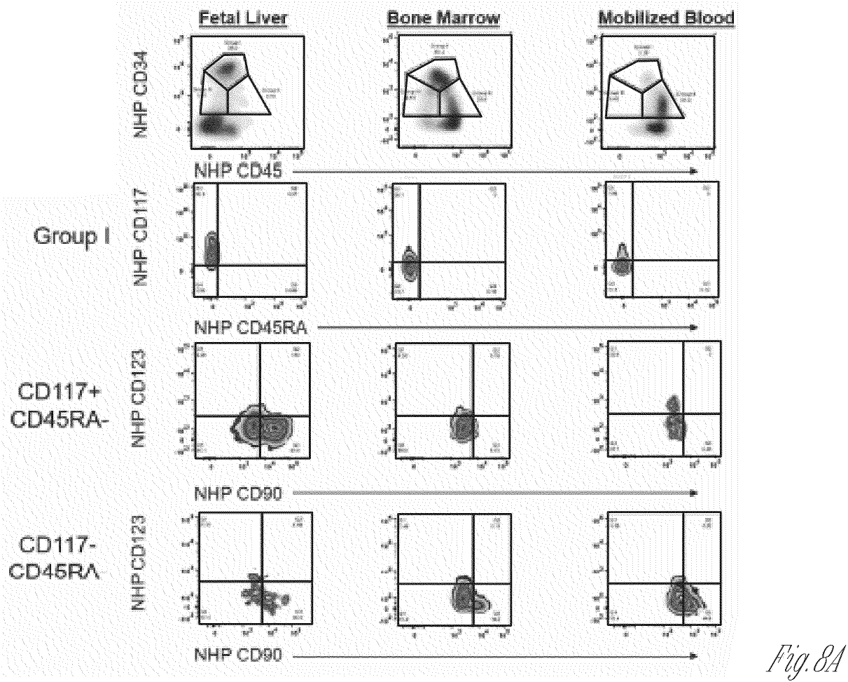

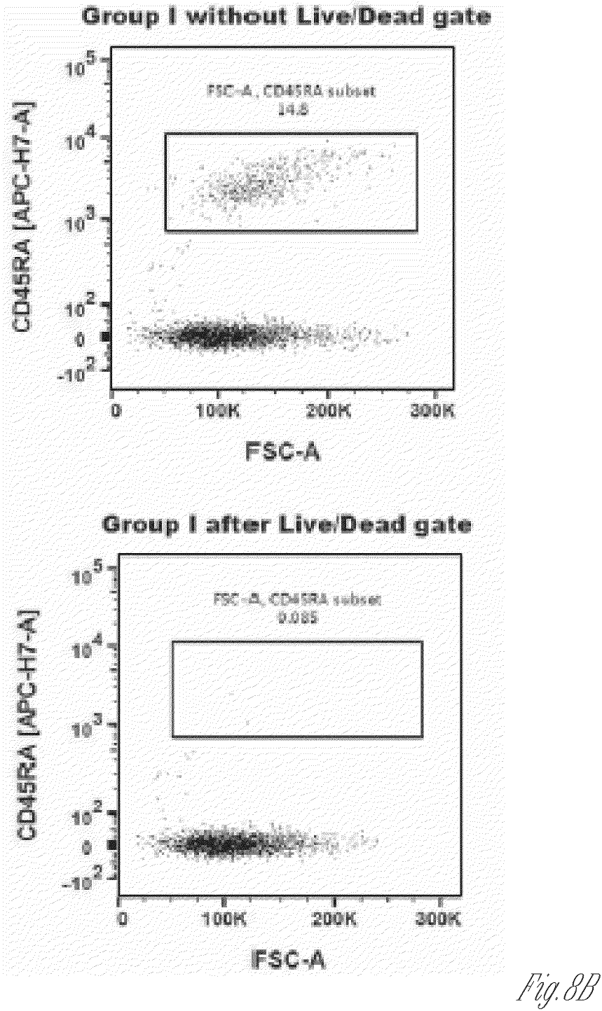

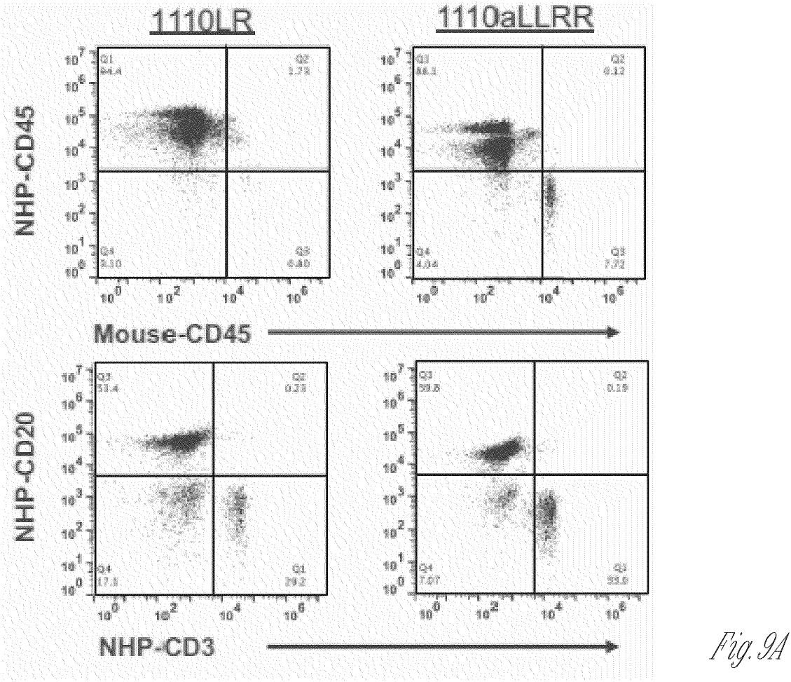

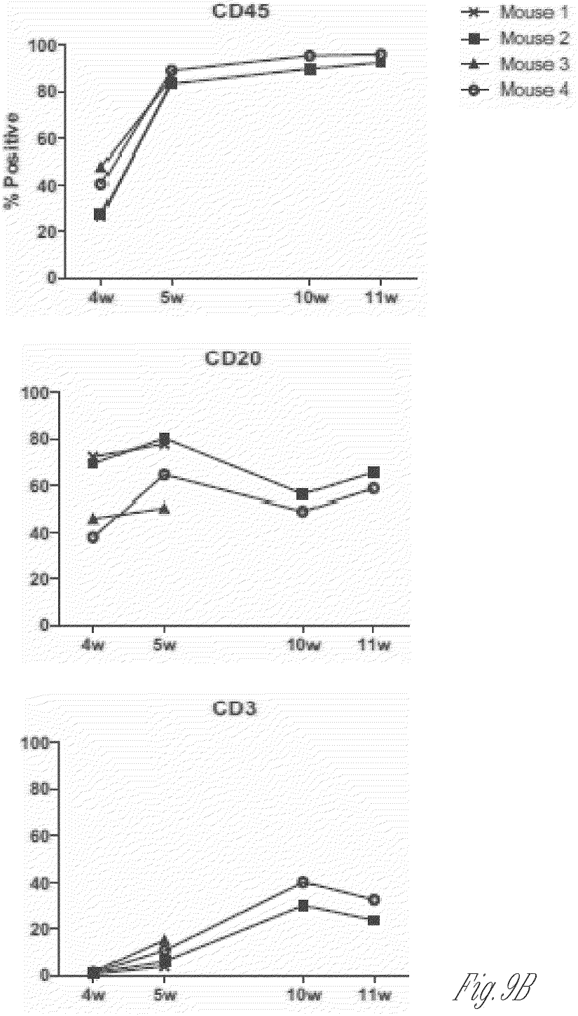

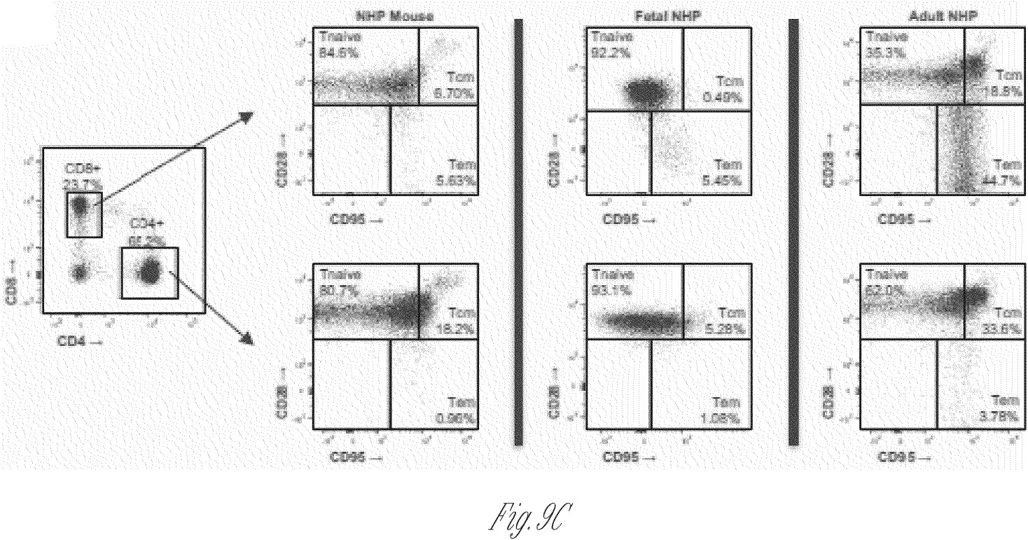

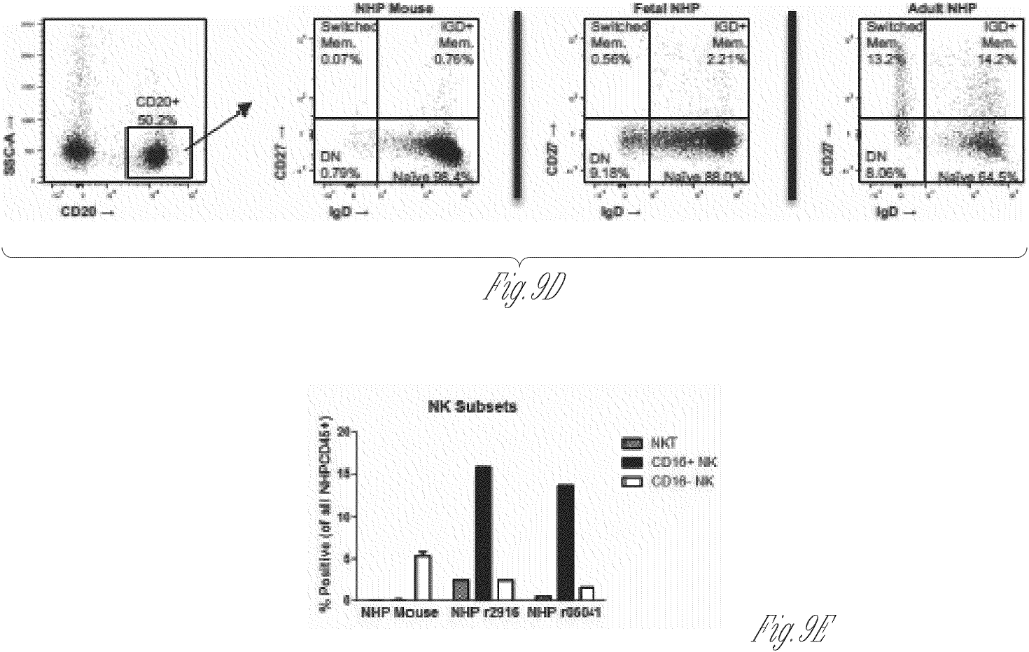

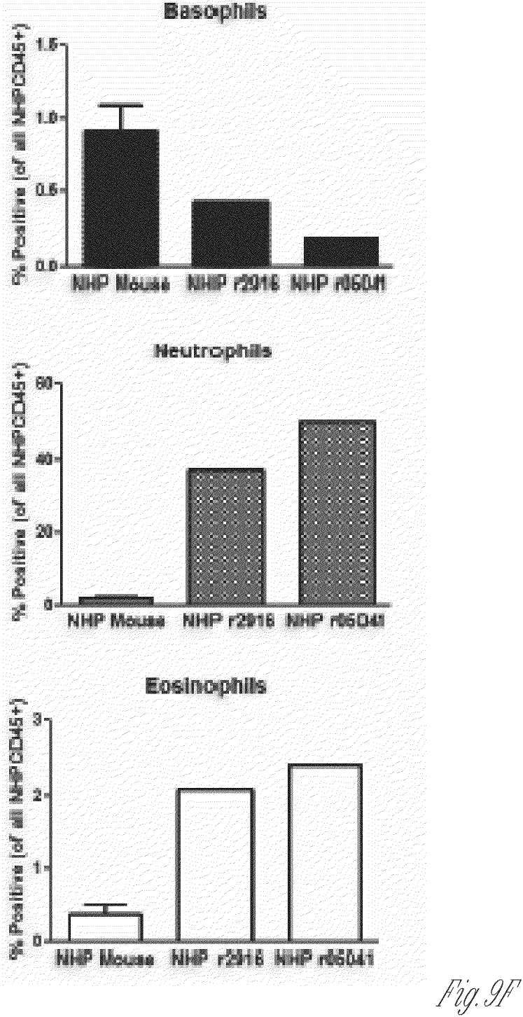

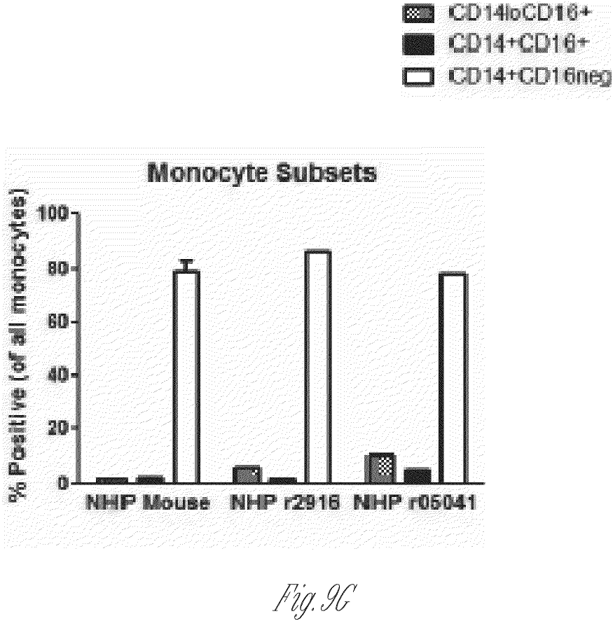

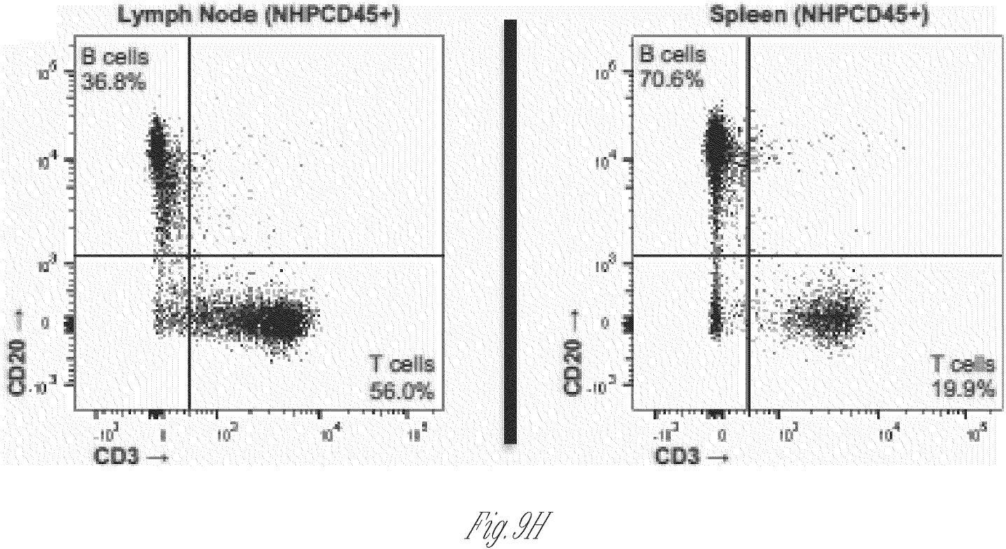

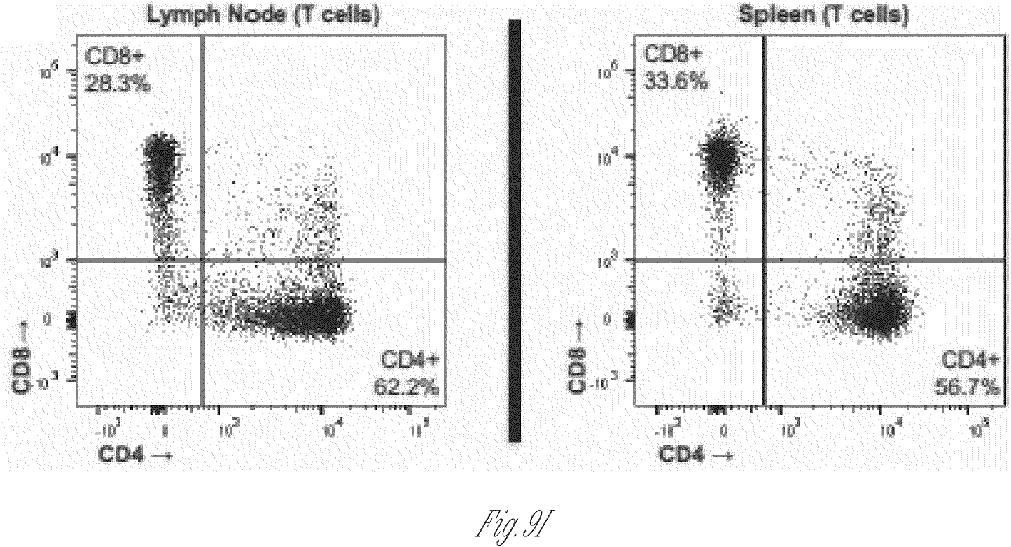

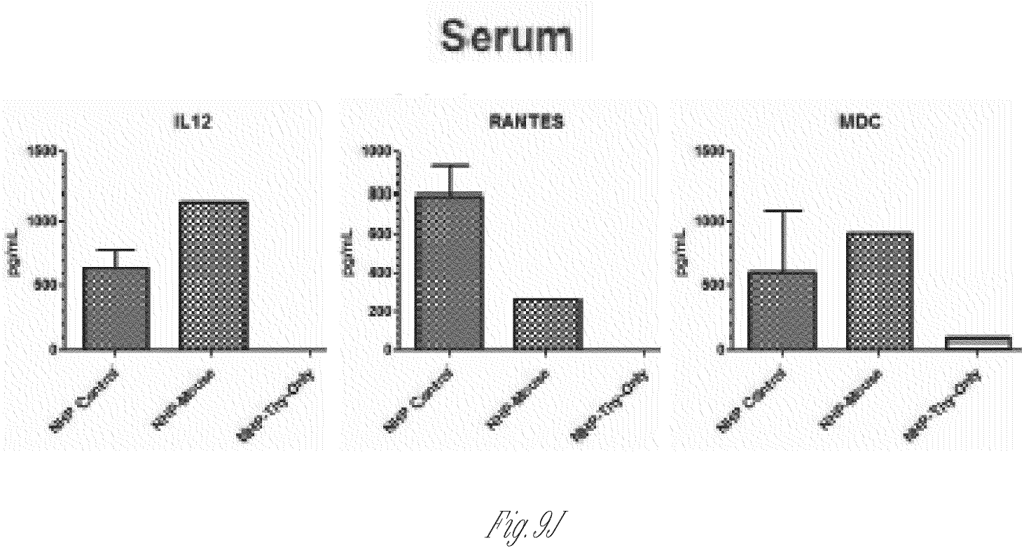

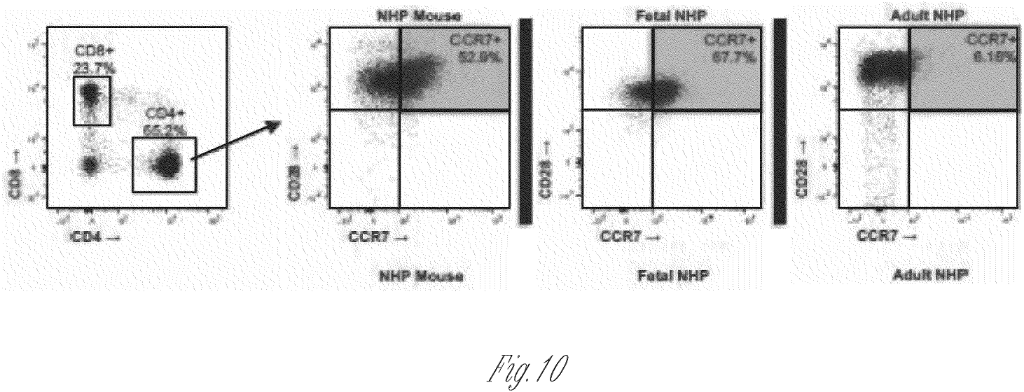

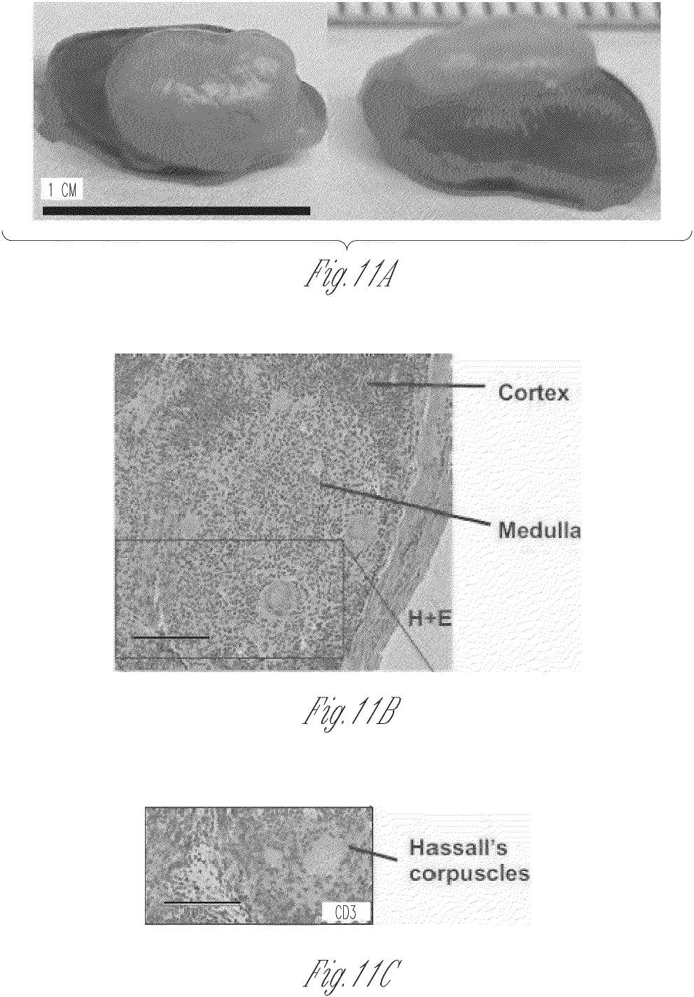

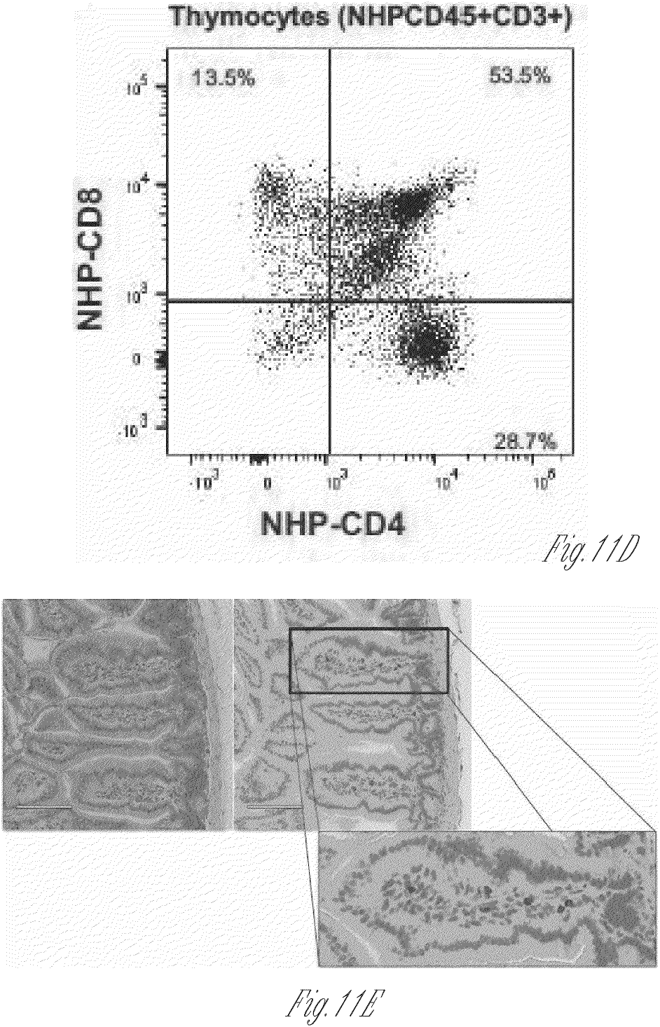

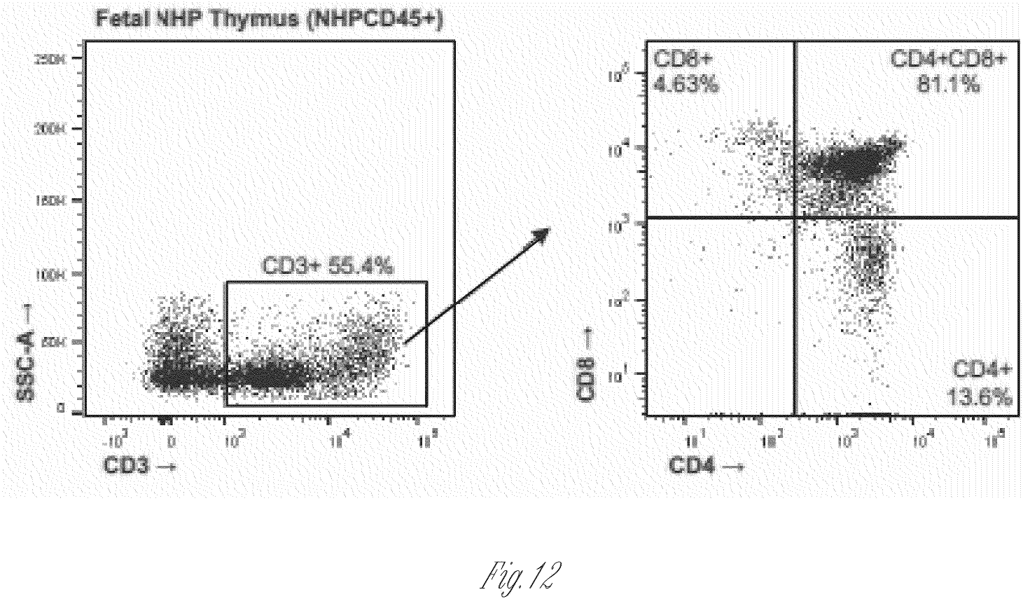

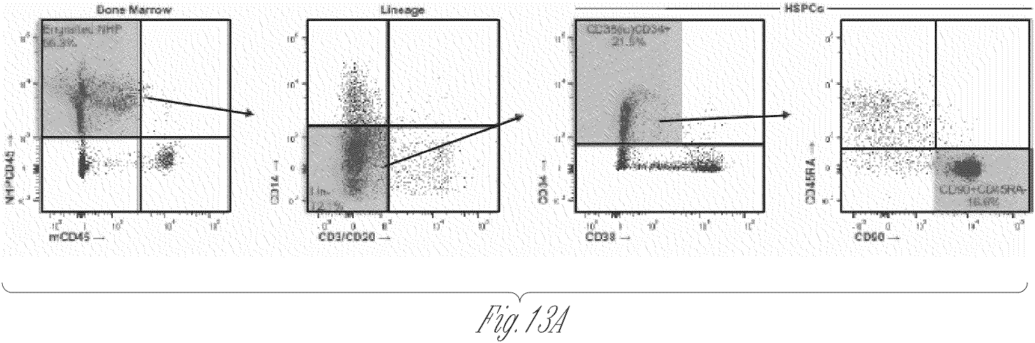

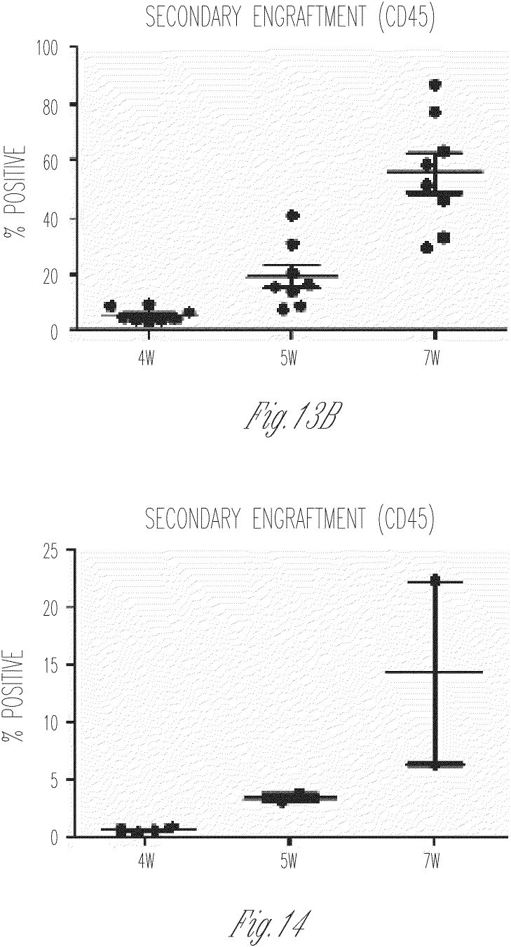

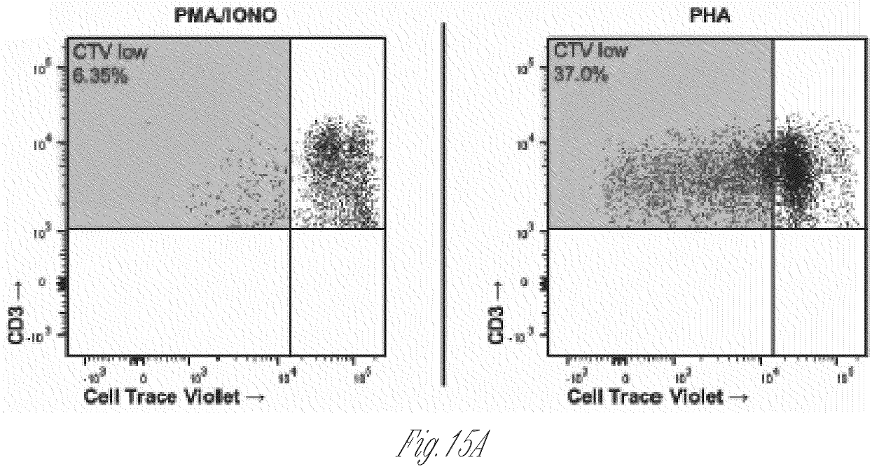

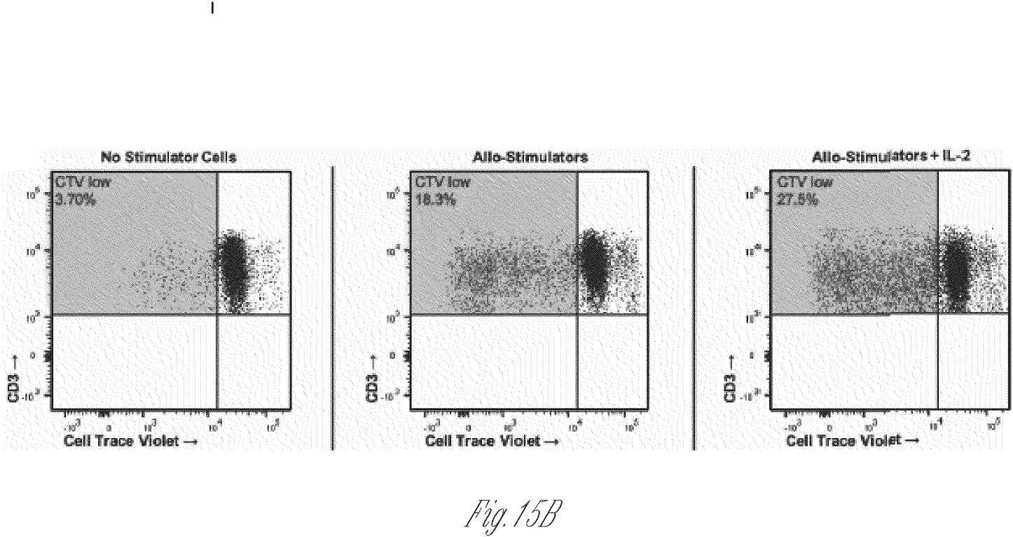

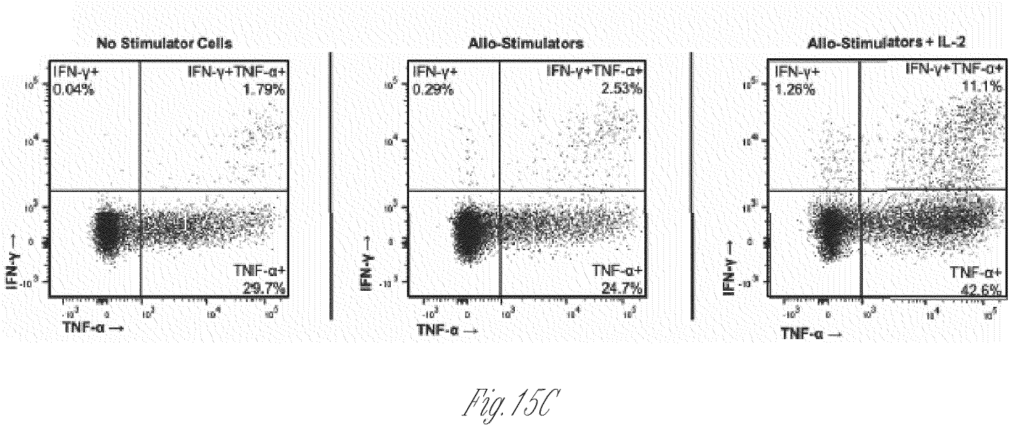

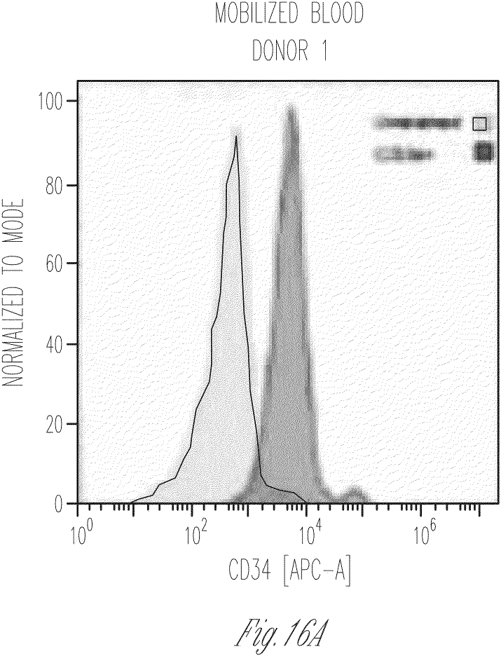

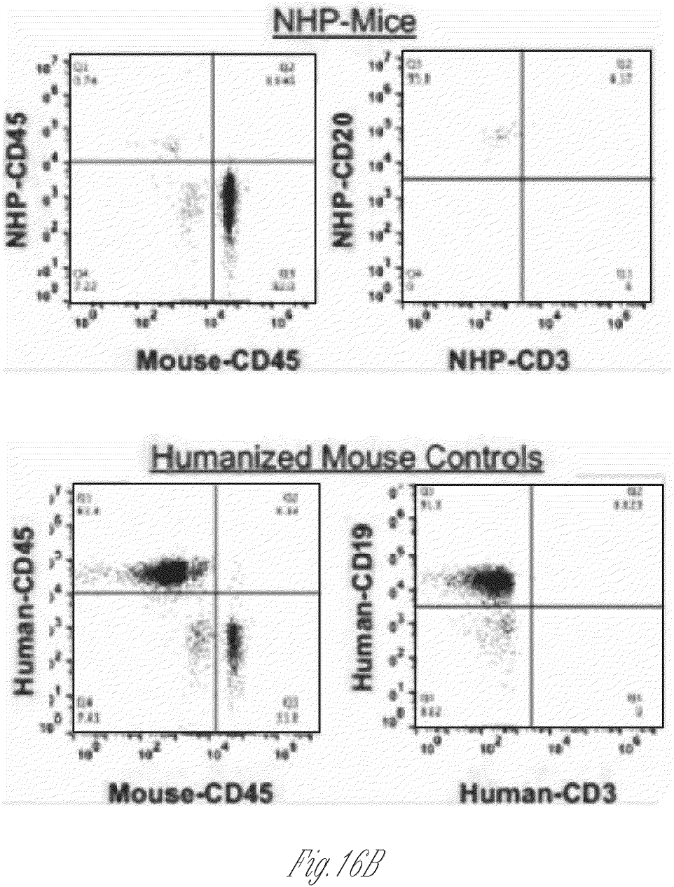

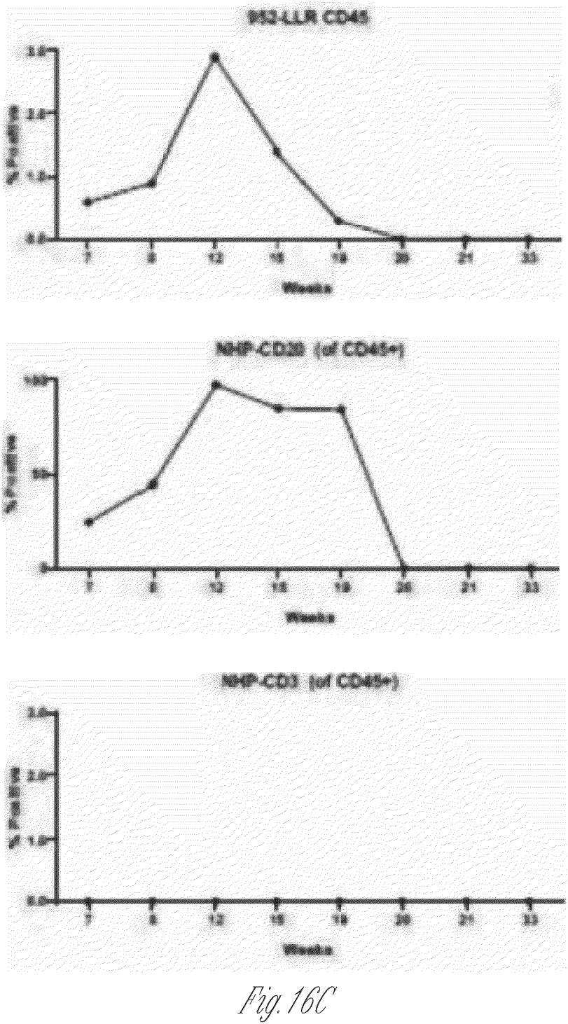

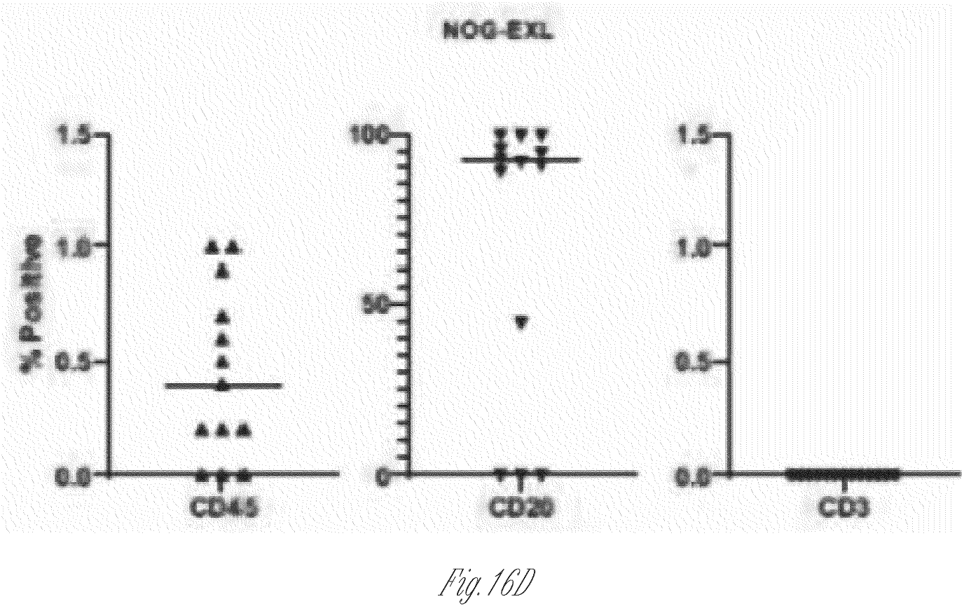

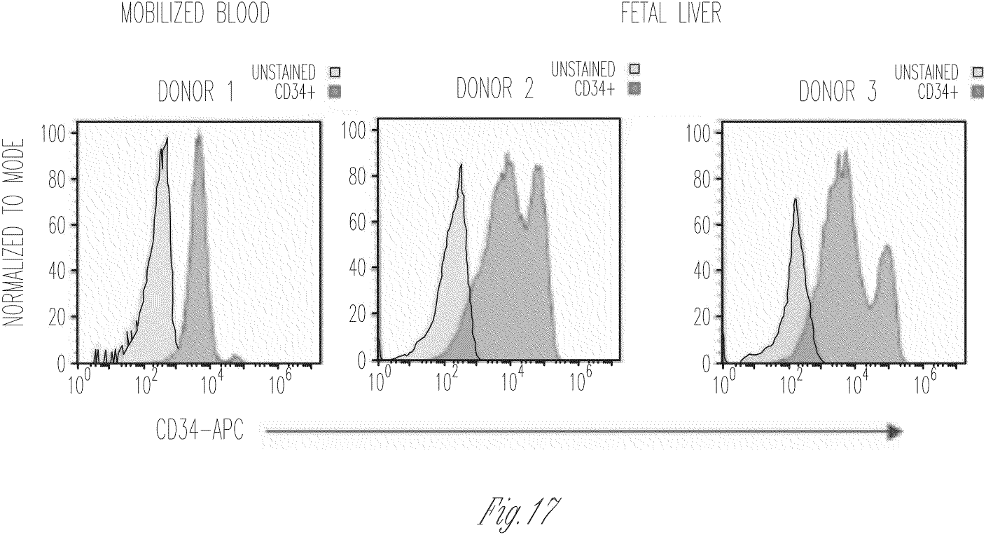

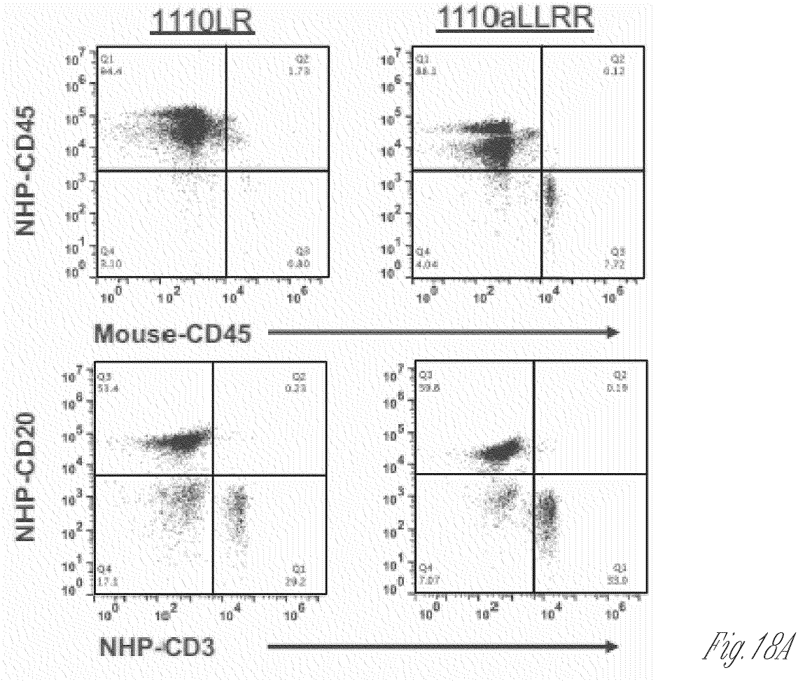

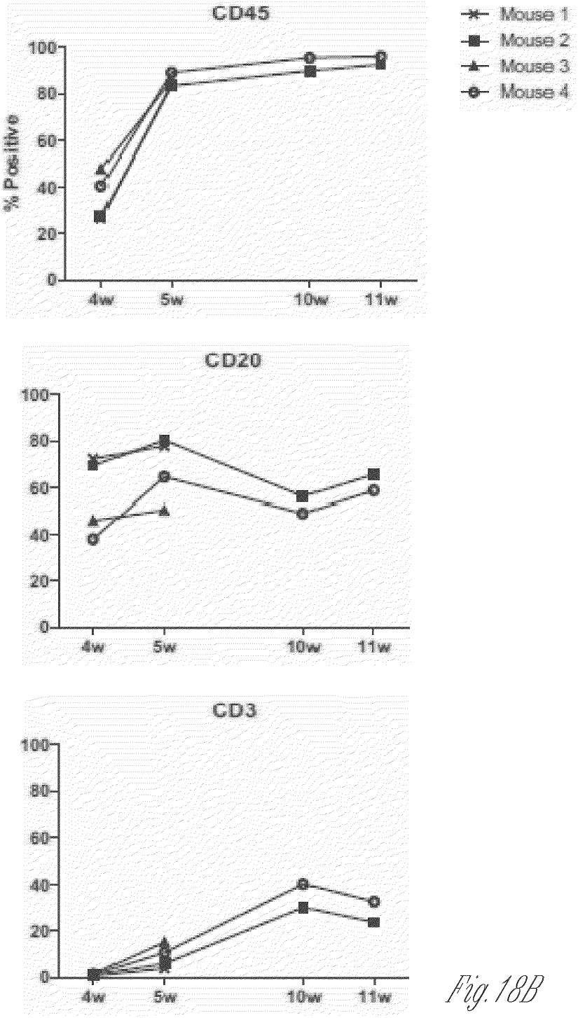

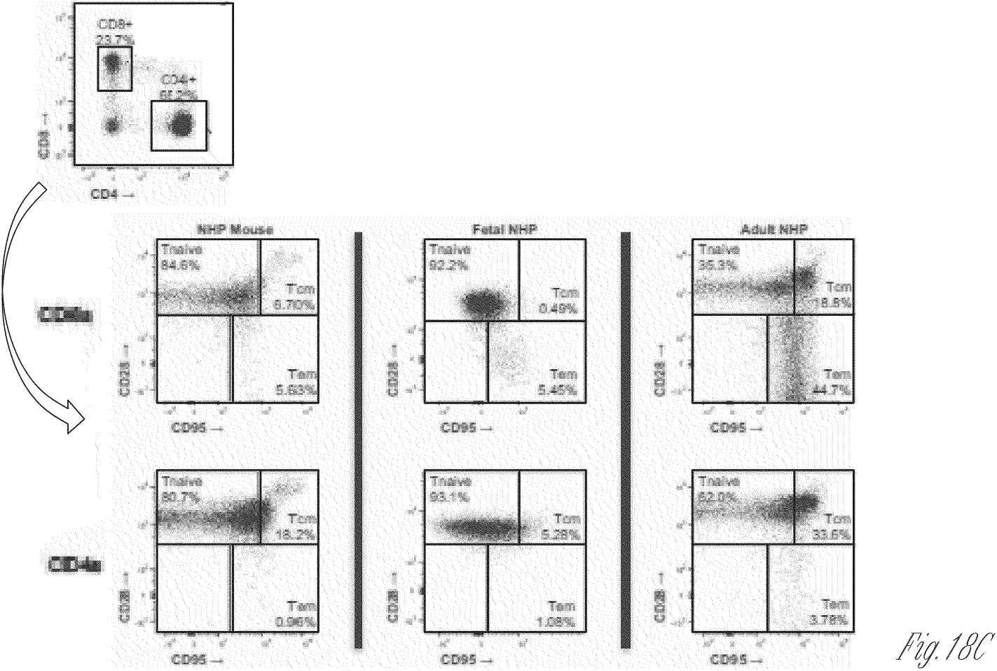

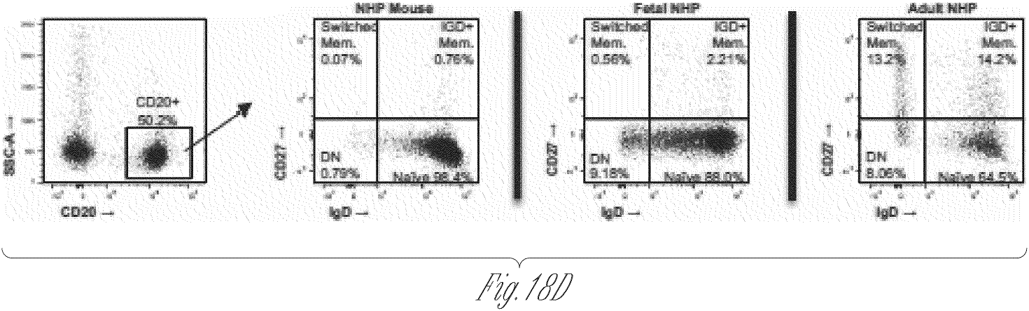

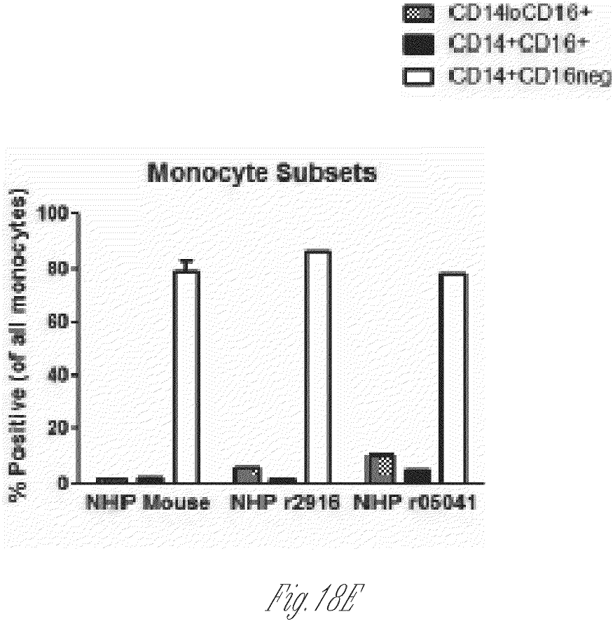

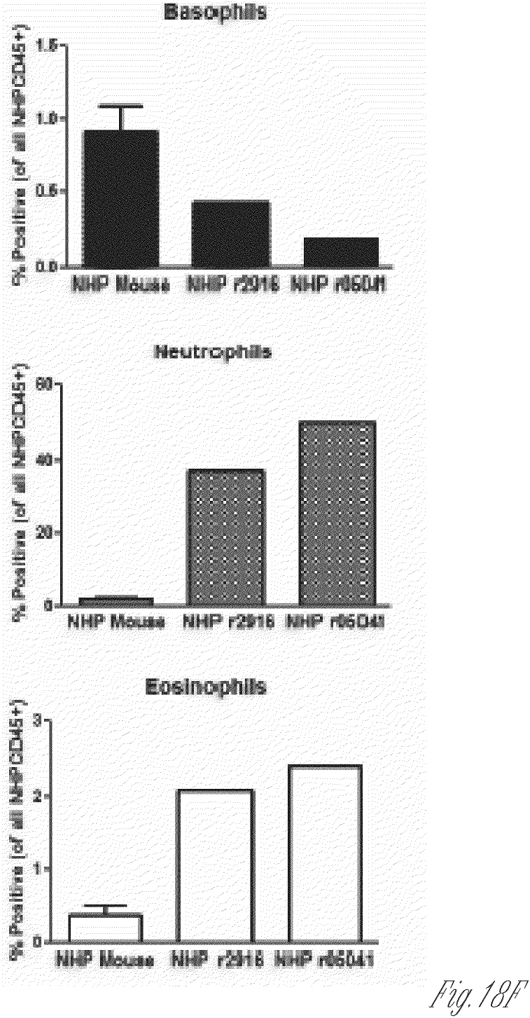

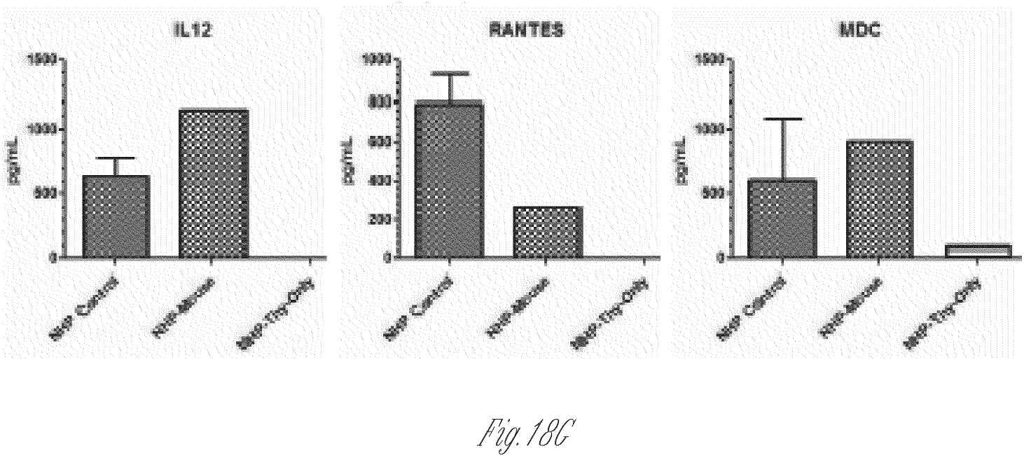

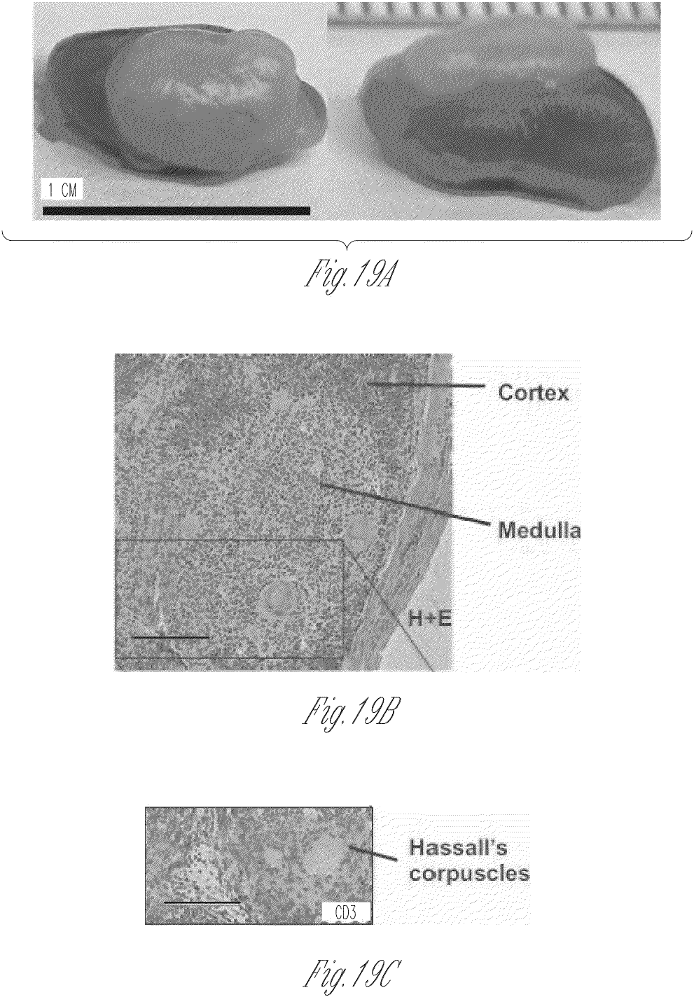

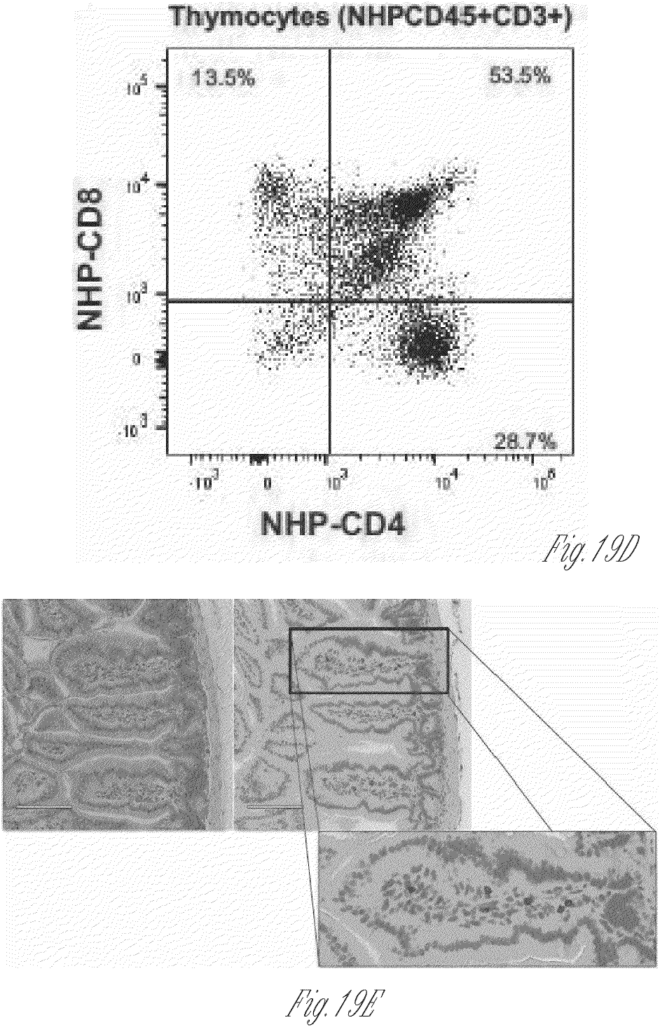

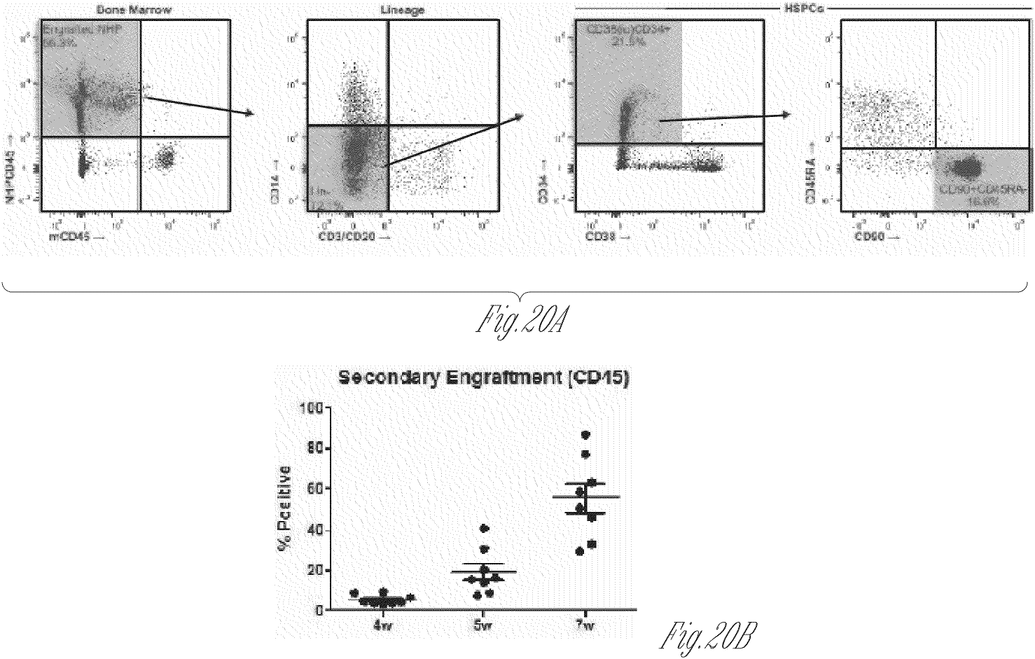

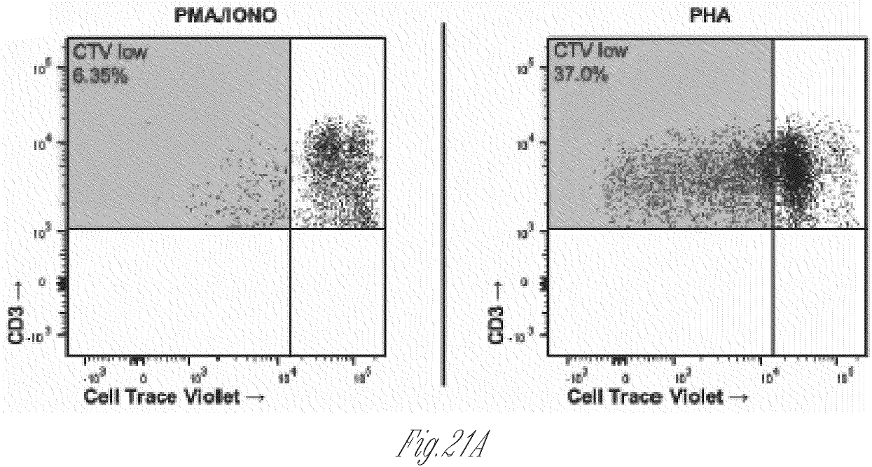

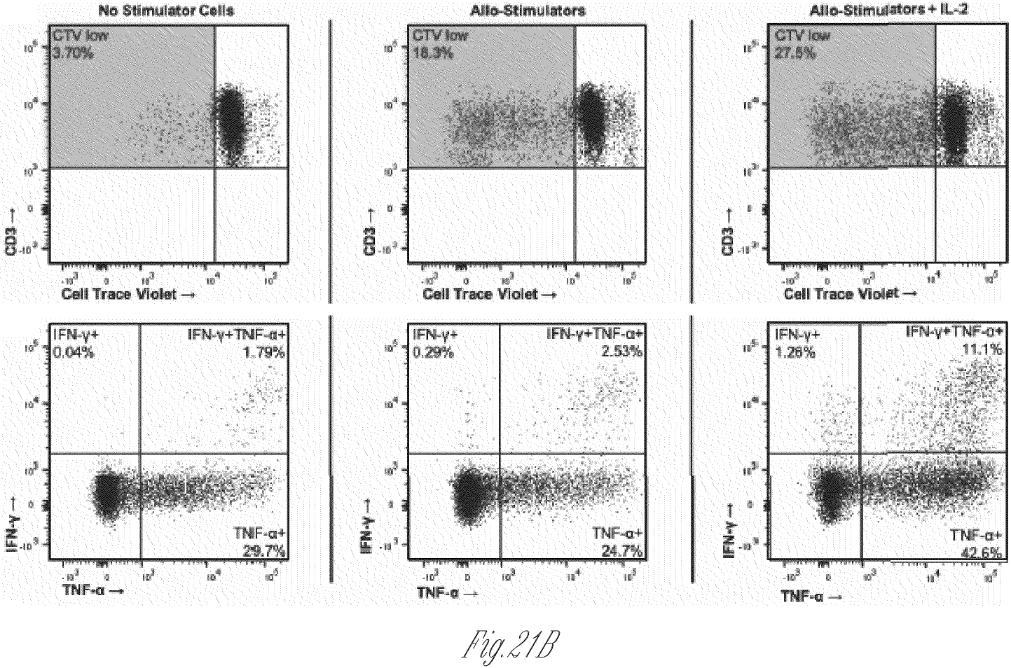

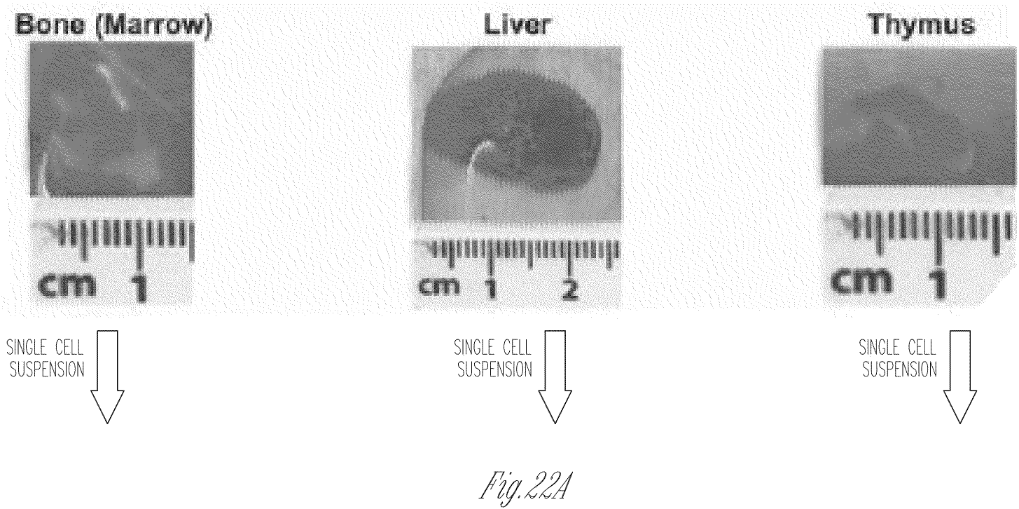

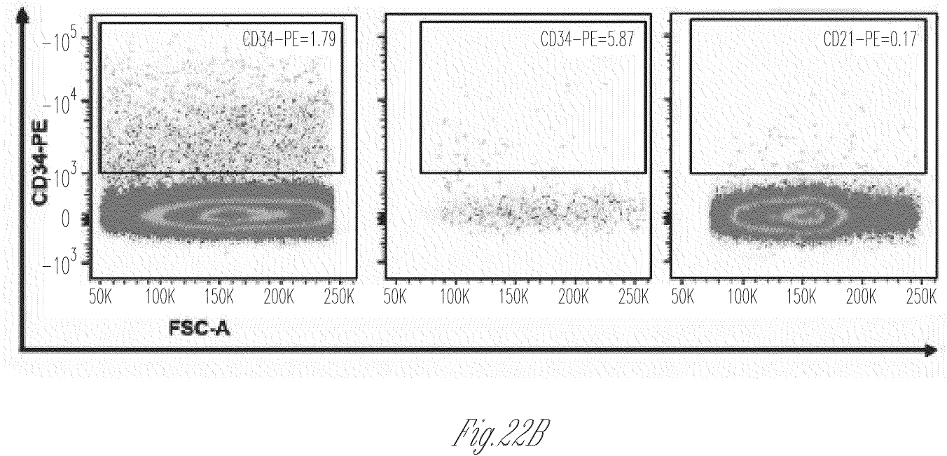

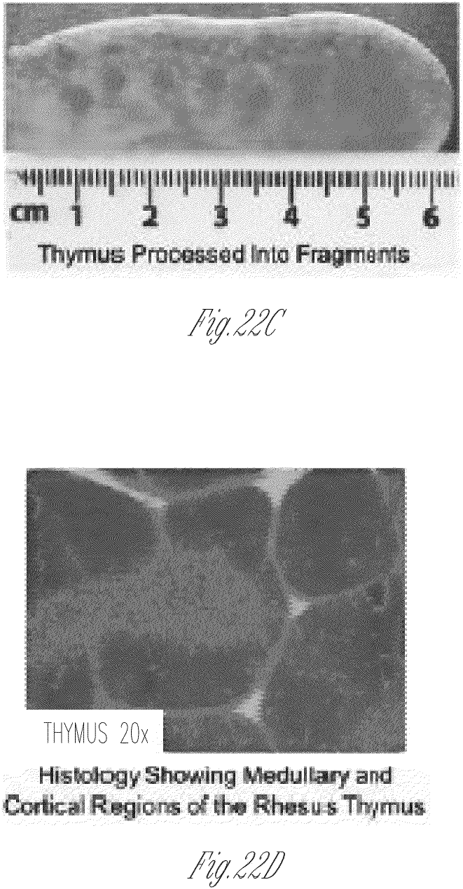

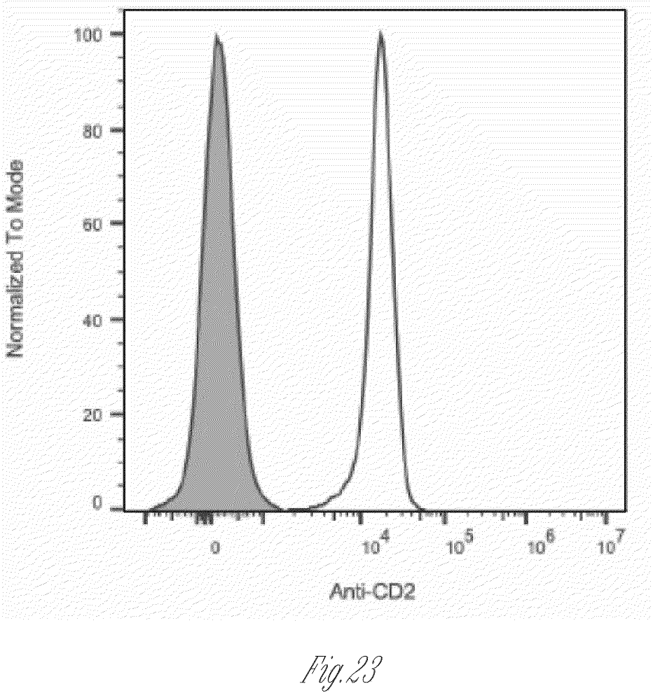

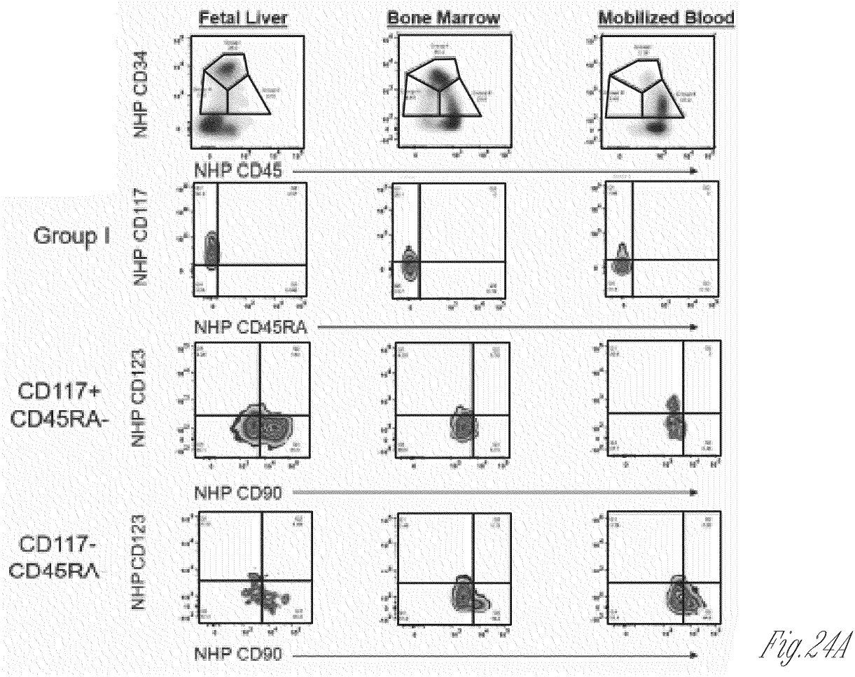

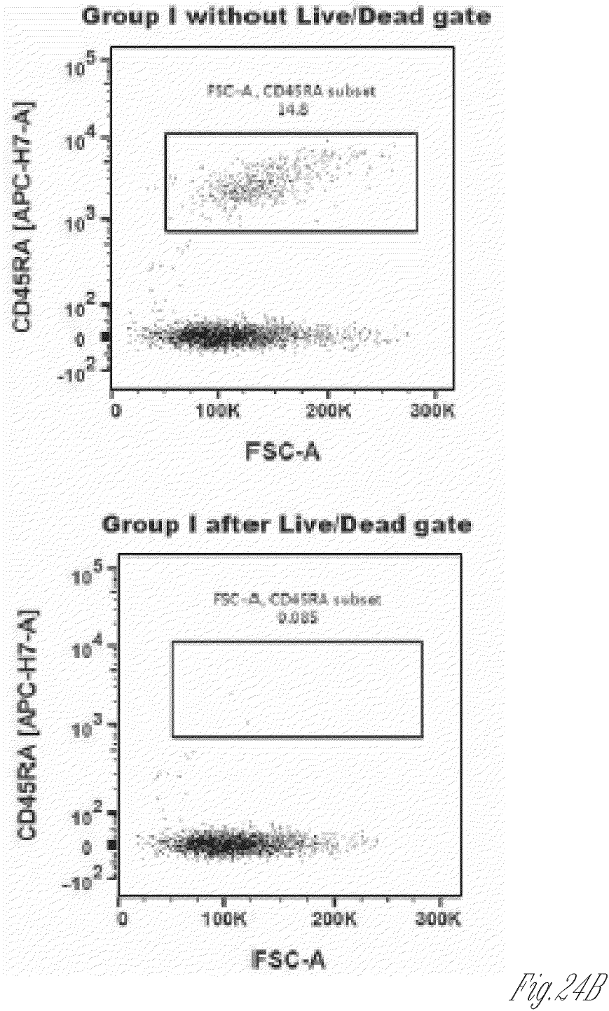

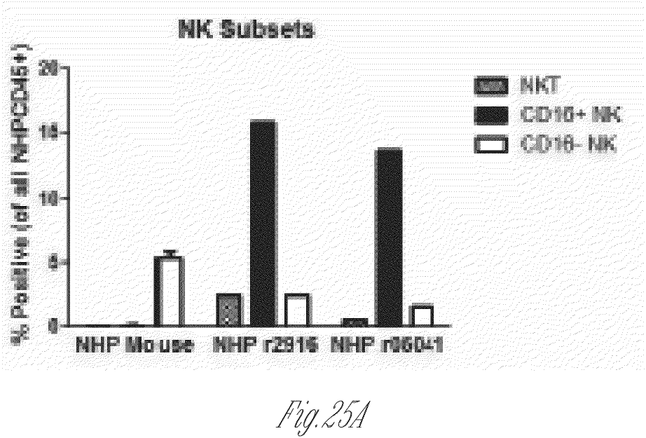

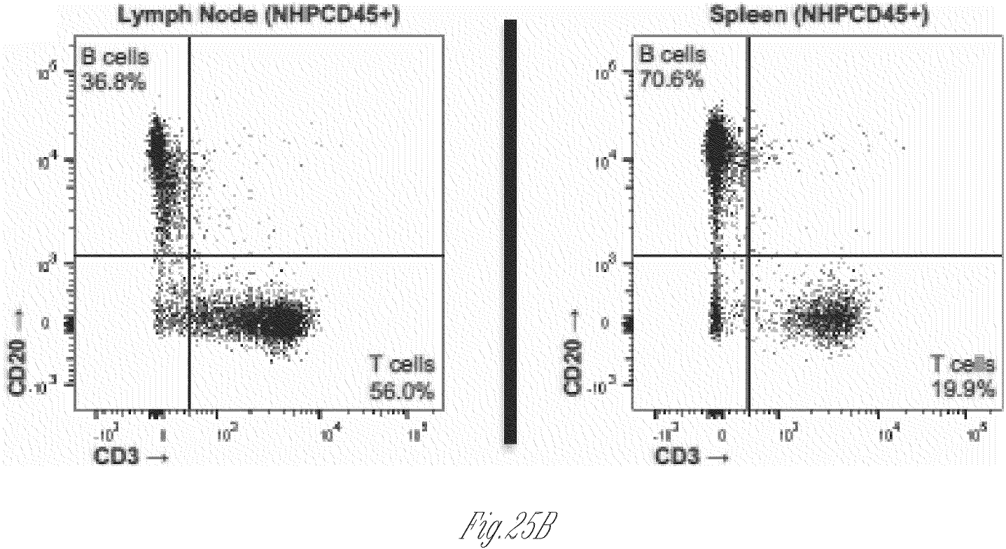

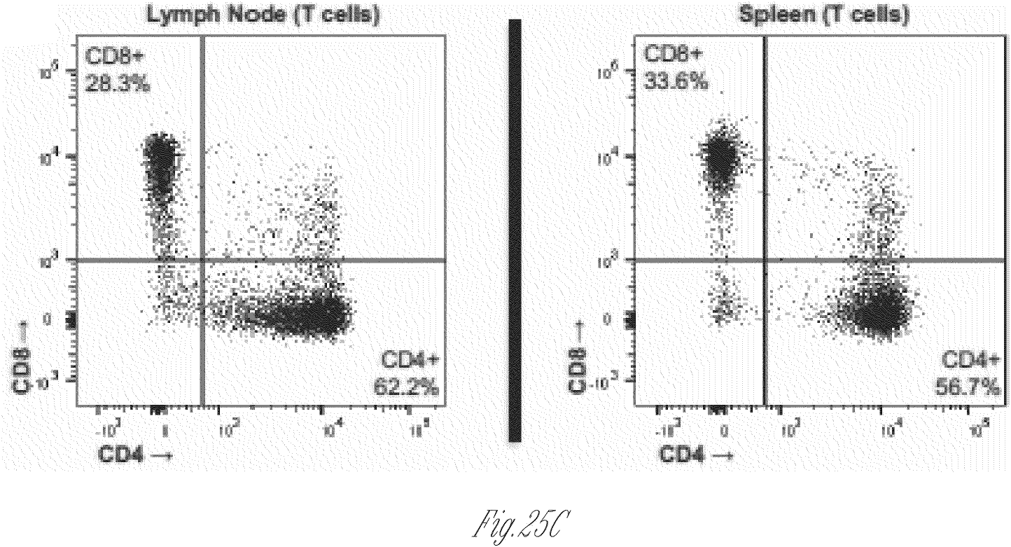

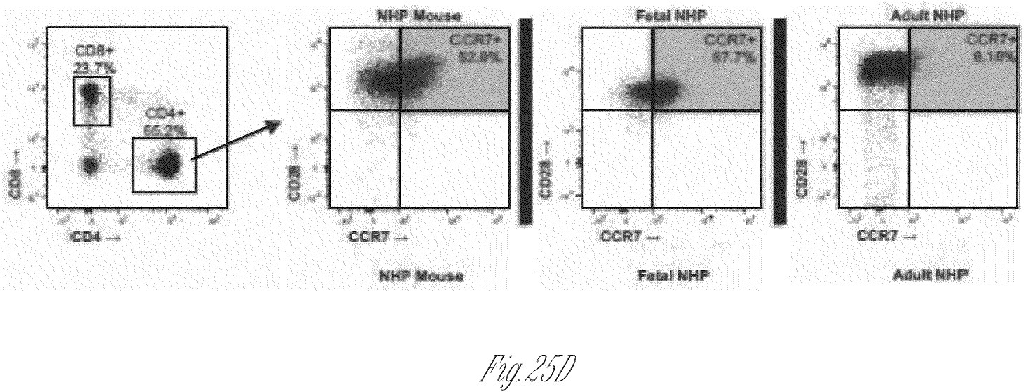

Injection of NHP hematopoietic stem cells (HSCs) into immune-compromised NOD.Cg-Kit^{W-41J} Tyr⁺ Prkdc^{scid} II2rg^{tm1Wjl}/ThomJ (NBSGW) (McIntosh et al., 2015) mice, combined with surgical implantation of NHP thymus fragments (for in vivo MHC-specific T cell selection), resulted in durable engraftment of primate immune cells, although the efficiency was lower than in the NOG-EXL and NSG-SGM3 lines. . Such an in vivo model is useful in studies of therapeutic interventions where T cells play a central mechanistic role. T cell-centric data from primatized mice (e.g., assessing the effects of multiple therapeutic interventions on simian immunodeficiency virus viral load in CD4+ T cells) allows for larger cohort sizes and lower animal numbers in downstream full-scale NHP studies (FIG. 1).

Additionally, a primatized mouse model could have a significant positive impact by increasing the rigor and reproducibility of experiments. Animal-to-animal variability due to genetic variation, especially with regard to major histocompatibility complex (MHC) type differences, makes it difficult to discern the effect of therapeutic interventions vs random background differences among individuals. The inbred Mauritian cynomologus macaque (MCM) population is an extremely useful preclinical model in this regard, as almost all of the MHC diversity in these animals is restricted to six high-frequency MHC class I and class II haplotypes (Wiseman et al., 2009). Haplotyped MCMs allow for the opportunity to investigate in vivo immunology mechanisms in an MHC-controlled model.

Due to high costs, NHP research tends to be performed by only well-established investigators with deep financial resources. To purchase, house, and provide veterinary care for one adult rhesus macaque (i.e., one data point) for a 6-month experiment the price is approximately $8000-$12,000 (internal estimates).8 Depending on experimental design, the use of NHPs to determine statistically significant differences between treatment groups of three or more animals per group can generate animal costs in the hundreds of thousands of dollars.

The quality of preclinical trials can be diminished if statistical significance is not achieved and/or promising results are not confirmed by (costly) replicate experiments. In turn, ambiguous NHP data can result in failure of downstream clinical trials, or prevent the clinical evaluation of promising therapies altogether. The present model enables replication of experimental data on the scale of potentially n ≥ 100 per cryopreserved tissue set, which is not feasible in whole-animal studies, even when using haploidentical MCMs.

To date, researchers have demonstrated that NHP blood cells from rhesus macaques (Larochelle et al.. 2011) and baboons (Huang et al., 2017) can be engrafted into immune-compromised mice, akin to early iterations of humanized mice, e.g., PBL-hu mice (Mosier et al., 1988). BLT/NeoThy type humanized mice are an improvement over PBL-hu models, specifically because they overcome the graft-vs-host disease and short experimental windows associated with PBL-hu models (Kalscheuer et al., 2012; McIntosh & Brown, 2015)), Similarly, a BLT/NeoThy type primatized mouse model would have clear advantages over primatizing mice with NHP peripheral blood alone. Indeed, the present model is in line with the criteria described in a recent review article about the potential value of primatized mice (Maufort et al., 2018).

### Approach

### Evaluate NHP T Cell Phenotype and Function in Fetal, Neonatal and Adult Animals

To gain an understanding of the baseline NHP immunity, the phenotype of representative T cells in colony animals is assessed, since T cells are the type that is best modeled in humanized mice and likely in primatized mice. The functionality of the T cells are assessed in in vitro mixed lymphocyte reaction (MLR) assays to further document the naturally occurring biology that we are seeking to model. These data are a reference. The experiments focus on animals at different ontogenetic stages, e.g.. in investigating how aging impacts the T cell repertoire composition and function.

Research Design. The experiments utilize freshly obtained and/or frozen NHP peripheral blood from Herpes B negative donor animals. Blood from fetal, neonatal, and adult rhesus macaques that optionally have been MHC typed for presence/absence of the A01 haplotype via flow cytometry (Holman et al., 2017) are employed. Adult MCM peripheral blood, from animals that have been haplotyped by DNA sequencing, is also used. Blood is collected from n=3 of the above-mentioned groups for a total of 12 animals. If possible, animals of a consistent sex, haplotype and blood group type are used. Blood cells are processed and separated as previously described in Brown et al. (2018) and Maufort et al. (2018). A portion of all cells are cryopreserved (e.g., for obtaining RNA for differential gene expression studies). Cells are stained and analyzed via flow cytometry for surface and intracellular markers associated with naïve, memory, various effector subtypes, and regulatory cells. Additionally, myeloid and non-T cell leukocyte markers are used to profile the composition of accessory cells, e.g., antigen presenting cells required for in vivo T cell function. Cells are then tested in MLR assays with target cells from haplomatched and haplomismatched animals, with PHA-stimulated positive controls, looking for differences in CFSE dye dilution, i.e., cellular proliferation (FIG. 3). MLR culture supernatants are frozen for later studies of cytokine profiles and their associations with effector subtype mechanisms of action. All experiments are conducted in a BSL level 2 biosafety cabinet as a precaution against zoonoses.

Samples from each of these groups have distinct profiles of effector and regulatory T cells, and these differences are consistent in replicate experiments. Like previously published works comparing human fetal and adult blood (Mold et al., 2011), an increased frequency of regulatory T cells is observed in fetal NHP blood vs adult. Accordingly, MLR cultures with fetal samples show a general decrease in T cell proliferation vs haplomismatched targets due to a higher proportion of regulatory T cells in the cultures.

### Primatize Immune-Compromised Mice with NHP Hematopoietic and Lymphopoietic Tissues

Creation of a primatized mouse model using a BLT/NeoThy approach would have significant value for generating data that could inform downstream NHP studies. This in turn could reduce costs of this type of research, as well as open up new opportunities for studying age-related changes in immune cell phenotype and function (Simon et al., 2015).

The approach to making primatized mice has some similarities to the approach in Brown et al. (2018), which describes the creation of the NeoThy model (using neonatal human tissues) in comparison with fetal tissue controls. Preliminary data indicate that a large population of CD34+ cells is present in adult rhesus bone marrow (data not shown) and in fetal bone marrow and liver (FIG. 4). These tissues are harvested for HSCs, as has been described in Hu-mice (Lan et al., 2006; Kalscheuer et al.. 2012). Preliminary experiments demonstrated the feasibility of obtaining anatomically normal thymic fragments from NHP necropsies. Tissue from fetal (about 100 days gestation), neonatal, and adult rhesus and from adult MCMs (n=3 for each group) is processed as previously described and cryopreserved for downstream primatization experiments (Brown et al.. 2018). For all ages of animals, bone marrow, thymus and blood are obtained. For fetal animals, liver is also obtained, as this is the predominant site for hematopoiesis (and thus a source for HSCs) during fetal gestation. NOG-EXL, NSG-SGM3 and NBSGW immune-compromised mice are i.v. injected with 1-5×10⁵ CD34+ HSCs. Additionally, a thymus fragment from the autologous or haplomatched donor is surgically transplanted under the mouse kidney capsule. Surgeries and monitoring for primate immune cell engraftment are conducted similarly to published methods in Brown et al. (2018). Samples are analyzed at multiple time points for up to four months for T cell and other immune markers, retained for histology, and blood and tissue will be cryopreserved for future differential gene expression studies.

Primate CD45+ immune cells are observed in the circulation of primatized mice beginning around week 4 to 8 post-surgery. These numbers at first are mainly B cells, but around weeks 10-12 T cells are detectable. By week 16, a full complement of T cells and accessory immune cells are detected in the blood, spleen, and various tissues in the mice. There is a lack of graft-vs-host-disease in the animals compared with mice injected with NHP peripheral blood alone, i.e., a primate PBL-mouse.

Alternatively, injections of peripheral blood from specific NHP donors rather than using HSCs and thymus tissue are used for primatization. HSCs may be treated with Stem Cell Factor and/or other factors/cytokines to increase their engraftment potential.

This has implications for the establishment of fetal-maternal tolerance and for age-associated differences in the ability to effectively clear viral infections (Burlingham et al., 1998; Nguyen et al., 2017). Detecting species-specific differences in viral tropism, or the ability for an infectious agent to jump from infecting monkeys of one species to infection of another species, or going from monkeys to humans, will also be of interest.

### EXAMPLE 2

### Materials and Methods

### Cellular Immunoprofiling

Retro-orbital bleeds of the primatized mice were performed at multiple timepoints, from which PBMC was isolated for flow cytometric analysis of T cell. B cell, and monocyte subsets and activation. Cells were stained for nhpCD45 (clone D058-1283), CD45RA (clone 5H9). CD3 (clone SP34-2). CD8 (clone RPA-T8). CCR7 (clone 150503), CD4 (clone L200), CD28 (clone CD28.2). CD20 (clone 2H7), CD14 (clone M5E2), IgD (clone L200) (BD Biosciences, San Jose, CA), moCD45 (clone 30-F11), CD95 (clone DX2), CD27 (clone 0323), CD16 (clone 3G8), HLA-DR (clone L243), CD90 (clone 5E10) (Biolegend, San Diego. CA), and CD38 (clone OKT10, Caprico Biotechnologies. Norcross, GA). Upon necropsy, fresh splenocytes and lymph node tissue were also isolated and stained with the aforementioned antibodies. Unprocessed bone marrow collected from long bones at time of necropsy was stained for CD38, moCD45, CD45RA. CD20, CD14, CD90, CD3 (clones listed above), and CD34 (clone 561. Biolegend, San Diego, CA).Cylometric data was acquired using an LSR II FACS machine (BD Biosciences, San Jose, CA). Data analysis was performed using FlowJo software (Treestar, San Carlos, CA).

### Mixed Lymphocyte Reactions and Stimulation Assays

Fresh isolated splenocytes from primatized mice were collected and labeled with Cell Trace Violet (CTV) (Thermo Fisher Scientific, Watham, MA) at a concentration of 1 uL CTV per 1×10⁶ cells. These responder cells were then plated at 200,000 cells per well in RPMI + 10% FBS growth media for 5 days under various stimulatory conditions. Allogenic MLR was set up utilizing irradiated, Cell Trace Far Red (CTFR) (Thermo Fisher Scientific, Watham, MA) labeled adult NHP PBMC at a ratio of 2:1 responders to stimulators. Recombinant human IL-2 (PeproTech, Cranbury, NJ) was added to a subset of MLR wells at a concentration of 100 ng/well. Non-MLR stimulatory assays were similarly performed with 200,000 CTV-labeled stimulator cells per well. Phorbol 12-myristate-13-acetate (PMA; 810 nM) with ionomycin (13 µM) (Biolegend, San Diego, CA) or phytohaemagglutinin-L (PHA; 10 µg/mL) (Thermo Fisher Scientific, Watham. MA) were added to each well prior to a 5-day incubation period. Cells were then re-stimulated with PMA (81 nM) and ionomycin (1.3 µM) with Brefeldin A (5 µg/mL) (Biolegend, San Diego, CA) for 5 hours. Following re-stimulation cells were stained for nhpCD45. CD90. CD3. CD8, and CD4 (clones listed above). Intracellular staining was performed for TNF-α (clone MAb11, BD Biosciences. San Jose, CA) and IFN-y (clone B27. Biolegend, San Diego. CA). Cytometric data was similarly acquired with the LSR II cytometer and analyzed with FlowJo software.

### Results and Discussion

### Low-Level Engraftment of Adult HSPCs in Transgenic Immune-Deficient Mice

Engraftment of a commonly available NHP HSPC source, adult mobilized blood (AMb), was tested in an immune-deficient NSG-variant mouse host, the NBSGW. This variant was previously developed and utilized for successful engraftment of a human immune system in a mouse host (McIntosh et al., 2015, Brown et al.. 2018). CD34+ cells were sorted from AMb via magnetic bead isolation (FIG. 5A), which were then injected IV into naive NBSGW mice. Establishment, level, and durability of engraftment were monitored via flow cytometry of peripheral blood samples for the presence of NHP-CD45+ cells. Despite 18 weeks of monitoring, engraftment of primate cells after infusion of AMb product within this host was not detected (data not shown).

A next-generation, commercially-available host, the NOG-EXL, which incorporates two transgenic human cytokines (GM-CSF and IL3), both of which have a high degree of homology and subsequent cross-reactivity with NHPs, was then tested. Given that the NOG-EXL and similar models (e.g., NSG-SGM3, MISTRG and the like) have been shown to enhance human engraftment and chimerism, the human-NHP cross-reactive cytokines may be sufficient to enhance the engraftment potential of AMb HSPCs, which is supported by utilization of adult bone marrow-derived HSPCs within a transgenic murine host (Radtke et al.). IV injection of 1×10⁵-1×10⁶ CD34+ AMb cells resulted in moderately durable engraftment in irradiated NOG-EXL mice, though at significantly lower levels of chimerism compared to humanized mice made with umbilical cord-derived CD34+ HSPCs (FIGS. 5B-C). Engrafted NHP leukocytes demonstrated CD20+ B cell predominance with an absence of T cell chimerism (FIG. 5B, second plot from left, FIG. 5C, middle plot). These results were reproducible (FIG. 5D), indicating that stable engraftment of adult HSPC populations was possible. However, weak engraftment and lack of T cell development limits viability of this model for downstream hematopoietic studies or leukocyte functional assays. The paucity of T cells was attributed to the absence of thymic implantation, which is consistent with several reported studies, e.g., injection of CD34+ cord-derived HSPCs alone in humanization studies. Thus, an effective transgenic murine host was identified and synchronous thymic implantation for the development of a functional peripheral immune compartment is likely a factor in that success.

### Hematopoietic Phenotypes of Adult VS Fetal HSPCs

To enhance the applicability of the model, a more robust and representative primatized immune system within the NOG-EXL host was explored. Several sources of HSPCs, including adult bone marrow and fetal liver-derived cells (100 days gestation) in addition to the AMb described above, were investigated. CD34+ cells were magnetically separated and enriched to >90% purity prior to quantification and cytometric phenotyping. Fetal-derived product revealed a significant enhancement of the CD34(hi) subpopulation compared to AMb, which is known to be associated with augmented engraftment (FIG. 6). Further sub-analysis additionally revealed a higher percentage of lineage^{neg}CD34^{hi}CD45^{mid}CD117⁺CD45RA⁻CD90⁺ (group VII) cells, which are described by Radtke et al. as being the HSPC subset with the greatest hematopoietic potential (FIG. 7). Based on these findings, fetal-derived HSPCs were selected as the HSPC source for the induction of NHP chimerism in the transgenic host due to their significant CD34(hi) and group VII percentages. Importantly, at this developmental age there was a sufficient quantity of CD34+ cells, as well as a developed thymus, to allow for BLT/NeoThy-type primatization of multiple hosts from a single donor.

### Robust Primatization of Transgenic Immune-Deficient Mice

1×10⁶ fetal liver-derived CD34+ cells were intravenously injected into the irradiated NOG-EXL host and 1 mm × 1 mm of fetal thymus fragment was surgically implanted under the left kidney capsule after cell infusion (Brown et al., 2018). To facilitate removal of “passenger thymocytes” known to contribute to graft-vs-host-disease, the recipient mice were injected with human anti-CD2 antibody, which was demonstrated as having cross-reactivity in the fetal NHP (FIG. 8) (Kalscheur et al., 2015, Brown et al., 2018). Implementation of this protocol over two separate experiments yielded robust multilineage chimerism of NHP-CD45⁺ cells in all animals (n=6) (FIG. 9A). Engraftment was demonstrated in both CD20+ and CD3+ lymphocytes, with a significant increase in T cell percentages over time, indicating de novo T cell development via positive and negative selection in the thymic organoid (FIG. 10B). CD4⁺ and CD8⁺ cells were both present in the T cell compartment, with a majority of the cells demonstrating a naïve phenotype (CD28⁺CD95⁻), similar to the fetal NHP immune system. This is in contrast to the antigen-experienced NHP adult, which has higher relative percentages of central (CD28⁺CD95⁺) and effector memory (CD28⁻CD95⁺) T cells. (FIG. 9C) An interesting observation was made with regard to CCR7 expression on CD4+ T cells in primatized mice and primary NHP fetuses, which demonstrated marked enhanced compared to adult NHPs. CCR7 is known to be involved in thymic homing during murine immune development, raising the possibility that this upregulation could be related to tissue-specific chemotaxis and T cell maturation (FIG. 8). Also consistent with the fetal immunoprofile, the B cell compartment within the NHP mice was largely naive (CD27⁻IgD⁺), which was similarly less-mature than adult NHP controls (FIG. 9D). NHP-derived CD16^{neg} NK cells, (FIG. 10E) granulocytes (FIG. 9F), and monocytes (FIG. 9G) were also repopulated following engraftment. Furthermore, these animals developed comparable levels of the inflammatory cytokine, IL-12, as well as the chemokines, RANTES and MDC, to adult NHP controls (FIG. 9K). Taken together, these findings demonstrate the robust reconstitution of a diverse, multilineage immune system following hematopoietic engraftment. In contrast to prior murine primatization models, the multilineage nature of this engraftment has wide-reaching implications for the fields of stem cell physiology and immunologic maturation, especially within the context of fetal development.

### Fetal Thymic Organoid, Secondary Lymphoid Repopulation, and Tissue Resident Immune Cells

Similar to previous reports in humanized mice (Brown et al.. 2018), the transplanted fetal NHP thymus fragment produced a large thymic organoid within the kidney capsule (FIG. 11A). The histologic structure was similar to that seen in primary fetal thymus (FIG. 6, middle panel/right), with well-developed Hassall’s corpuscles (FIG. 11C) as well as CD3⁺ thymocytes within the cortical and medullary regions (FIG. 11B). Flow cytometric analysis of processed thymic organoid revealed developing double positive CD4⁺CD8⁺ thymocytes, as well as single-positive CD4⁺ and CD8⁺ T cells (FIG. 4D), which is similar to the immunoprofile of primary fetal thymus (FIG. 10). Importantly, secondary lymphoid tissues (lymph nodes and spleen) were also repopulated with NHP-derived lymphocytes as demonstrated by the presence of CD20+ B cells as well as CD4+ and CD8+ T cells.. Furthermore, NHP lymphocytes were found to be tissue resident within end organs involved in immune surveillance in defense, as demonstrated by histologic cross-sections showing lymphocyte aggregation and positive CD3 staining within the intestinal villi (FIG. 11E). Reconstitution of primary and secondary lymphoid organs are an essential component of immunologic maturation and function, which underscores the utility of this model for future immunologic studies involving acquired immunodeficiencies (e.g., simian immunodeficiency virus) and other lineage specific pathologies (e.g., Epstein-Barr virus). Furthermore, the presence of tissue resident lymphocytes opens this model to the study of diverse organ-specific disease processes involving immunologic defense and infectious pathophysiology.

### Bone Marrow Engraftment and Secondary Transplantation

To assess the efficiency of true hematopoietic stem cell (HSC) engraftment within the mouse bone marrow, primatized animals were sacrificed, bone marrow was isolated from the femur, and flow cytometric analysis of specific markers associated with HSC identify were conducted. As expected, the bone marrow contained a rich population of NHP HSPCs, including lineage-negative CD34⁺CD38^{lo}CD45RA^{neg}CD90⁺ cells (FIG. 13A). To test the viability and functionality of these HSPCs, total bone marrow was secondarily transplanted into two strains of transgenic immune deficient mice, NOG-EXL (FIG. 12) and NSG-SGM3 (FIG. 13B). The NSG-SGM3 variant includes transgenic human SCF in addition to IL-3 and GM-CSF present in the NOG-EXL strain. Importantly, secondary engraftment was evident in both mouse strains, demonstrating the strong engraftment potential of the transplanted HSPC subsets, e.g., indicating that at least one true multipotent hematopoietic stem cells (HSCs) engrafted. Interestingly, the addition of human SCF in the NSG-SGM3 model yielded a marked increase in secondary NHP-CD45⁺ chimerism. The utility of durable primary and secondary engraftment within these models is far-reaching and allows for future studies aimed at evaluating the hematopoietic potential of HSPC subsets and allows for testing non-myeloablative conditioning regimens prior to large animal implementation.

### Development of Functional Immune Cells

Having established the durability of engraftment and immune reconstitution, these animals were validated as a translatable model for functional NHP immunologic studies. A series of stimulation assays were performed to characterize the functional fitness of the peripheral T cell compartment. Splenocytes were exposed in vitro to known NHP mitogens (PMA/Ionomycin and PHA) to evaluate proliferative capacity of engrafted T cells. After 5 days in culture, stimulated T cells were found to have undergone robust, multigenerational proliferation, thus supporting their ability to respond to non-specific stimuli. Based on these findings, mixed lymphocyte reactions (MLRs) were set up with isolated splenocytes in an attempt to induce an MHC-restricted allo-specific immune response. Indeed, robust T cell activation and proliferation were induced in the presence of MHC-disparate irradiating adult NHP cells. This allogeneic response was further increased upon introduction the inflammatory cytokine, IL-2, which mimics the hostile post-allogenic transplant environment. Importantly, activated T cells produced IFNγ and TNFα in response to allogeneic stimulation, further demonstrating the development of functional T cells in the NHP mice. These findings have important implications in the field of transplant immunology, which is highly dependent on non-human primate studies given the disparate nature of small animal and human allo-immune responses. Moreover, the prohibitive cost of understudied human trials can result in allograft loss or patient death, further emphasizing the importance of effective large animal models. The uncovering of conserved allo-immune responses in these primatized mice, opens the door for extensive experimentation on a proven translational model, thus informing effective large animal studies and subsequent human implementation.

In conclusion, a durable primatized mouse model was established by transplanting fetal liver derived CD34+ HSPCs and thymic fragments into a cutting-edge and commercially-available transgenic murine host. Although there have been previous reports of mouse primatization, this represents the first model with robust multilineage chimerism and immune reconstitution within multiple physiologic compartments, mirroring that of the donor primate. This model is therefore poised to have wide-reaching applications within the fields of stem cell physiology, immunologic development, disease pathology, and transplant immunology, all of which are studied extensively within the non-human primate model. This primatized mouse is an efficient, cost-effective mechanism to inform implementation of novel therapeutic or diagnostic interventions in non-human primate studies, in transplantation and other contexts (e.g., virology), thus minimizing the risk and logistical burden of large animal models. By accelerating the pace of NHP studies through the utilization of this primatized animal, we hope that impactful and potentially life-saving approaches can be more rapidly translated to the bedside, thus efficiently advancing the field of clinical medicine.

### EXAMPLE 3

Non-human primates (NHPs) represent one of the most important models for pre-clinical studies of novel biomedical interventions. In contrast to small animal models, their widespread utilization is restricted by cost, logistics, and availability. We therefore sought to develop a translational primatized mouse model, akin to a humanized mouse, to allow for high-throughput in vivo experimentation leveraged to inform large animal immunology-based studies. As described herein, adult rhesus macaque mobilized blood (AMb) CD34+ enriched hematopoietic stem and progenitor cells (HSPCs) engrafted at low, but persistent, levels in immune-deficient mice harboring transgenes for human (NHP cross-reactive) GM-CSF and IL3. but not in mice with wild-type murine cytokines lacking NHP cross-reactivity. To enhance engraftment, fetal liver-derived HSPCs were selected as the infusion product based on an increased CD34(hi) fraction compared to AMb and bone marrow. Coupled with co-transplantation of rhesus fetal thymic fragments beneath the mouse kidney capsule, fetal liver-derived HSPC infusion in cytokine-transgenic mice yielded robust multilineage lymphohematopoietic engraftment. The emergant immune system recapitulated that of the fetal monkey, with similar relative frequencies of lymphocyte, granulocyte, and monocyte subsets within the thymic, secondary lymphoid, and peripheral compartments. Importantly, despite exhibiting a predominantly naive phenotype, in vitro functional assays demonstrated robust cellular activation to non-specific and allogenic stimuli. This primatized mouse represents a viable and translatable model for the study of hematopoietic stem cell physiology, immune development, and functional immunology in NHPs.

### Introduction

Humanized mouse models, created by grafting human hematopoietic stem and progenitor cells (HSPCs) into immune-deficient mouse hosts, have enabled important discoveries about human hematopoietic engraftment (Kamel-Reid et al., 1988) and leukocyte development (Mold et al., 2010). In recent years, humanized mice have become a useful tool for pre-clinical experimental study of human immune function (Baenziger et al., 2006). In a similar way, non-human primates (NHPs), such as the rhesus (Macaca mulatta) and cynomologous (Macaca fascicularis) macaques serve as important large-animal models for pre-clinical translational studies involving novel therapeutic or diagnostic interventions given their biological similarities to humans (Maufort et al., 2019). However, the high cost and prolonged timeline of acquisition, husbandry, and experimentation in NHPs is a critical barrier to their effective and efficient use in large-scale biomedical research.

A mouse model with an NHP immune system was developed to investigate in a high-throughput manner the relationship between NHP HSPC ontogeny stage and leukocyte engraftment and function. Successful development of such a primatized model would allow for investigation of the factors that influence NHP hematopoietic engraftment, and enable more efficient large-animal studies for transplantation immunology and virology research.

There have only been two prior published descriptions of NHP-derived hematopoiesis and leukocyte development in immune-deficient mice (Binhazim et al., 1996; Radtke et al.. 2019). However, there have been no reports of BLT/NeoThy-type primatiziation utilizing intravenous (IV) injection of HSPCs with surgical transplantation of thymus tissue, which is the current gold-standard for humanization studies. In their recent study. Radtke et al. utilized a powerful next-generation host, the MISTRG mouse, harboring multiple human hematopoietic transgenes, including SIRPα, M-CSF, Thrombopoietin, GM-CSF, and IL-3 on a RAG2-/- IL2Rg-/- background ((Rongvaux et al.. 2014). Despite their noteable success in inducing sustained hematopoietic engraftment, optimal chimerism relied on injection of a rare FACS-sorted HSPC population, resulting in a low-throughput model, complicating its feasibility for large-scale studies. Furthermore, although these primatized mice did develop T lymphocytes via the native murine thymus, these cells harbor limited functional capacity due to an absence of NHP thymic epithelium and concordant lack of NHP MHC selection cues during maturation. This resulted in a suboptimal model for translational characterization of MHC-restricted immune responses in NHPs.

These prior reports highlight the importance of synchronous tissue implantation coupled with cellular transfusion in an immune deficient host, importantly, with a sufficient transgenic background to allow for robust HSPC engraftment and host primatization. In our study, we aimed to develop a tractable BLT/NeoThy-type primatized mouse model to use in pre-clinical studies of ontogeny-associated hematopoietic engraftment, immunologic development, and leukocyte function.

### Materials and Methods

### Tissue Processing and Cell Purification

Experiments were approved by the Animal Care and Use Committee of the University of Wisconsin-Madison School of Medicine and Public Health. NHP liver was processed by macerating the tissue over a 100 µm cell strainer, and leukocytes were collected with Lymphocyte Separation Medium (Coming, Manassas, VA). HSPCs were enriched by using NHP-CD34-APC antibody (clone 563, BD Biosciences, San Jose, CA) and MACS anti-APC beads (Miltenyi, Bergisch Gladbach, Germany). NHP thymus was processed from fetal rhesus macaques by placing necropsied thymus in cold media, removing extraneous tissue, then dissecting into 1 mm × 1 mm fragments. Cells and tissue were cryopreserved in CryoStor CS10 medium (Stem Cell Technologies, Vancouver, BC Canada).

### Primatization Surgeries and Secondary Transplantation

Primatized mice were generated similarly to previous published humanization reports (Brown et al., 2018; Kalscheur et al., 2012). Briefly, 6-10 week old male and female NBSGW. NOG-EXL, or NSG-SGM3 mice were IV injected with 1 ×10⁵-1×106 CD34⁺ cells, and cryopreserved thymus fragments were surgically implanted under the mouse kidney capsule. Mice also received IV injection of αCD2 antibody (100 µg) at days 0 and 7 postsurgery, as previously described (Brown et al., 2018). All mice were treated with Buprenorphine SR at day 0, and Baytril antibiotic for 10 days post-surgery.

### Mouse Blood Collection and Immunoprofiling by Flow Cytometry

Peripheral mouse blood was sampled via retro-orbital bleed using heparin-coated capillary tubes (Thermo Fisher Scientifc, Waltham, MA) into microtubes containing 150 µl of 2% dextran/dPBS-/- and 150 µl of 0.5%-Heparin solution (Sigma Aldrich, Saint Louis, MO). After 20 minutes settling, the leukocyte-containing upper layer was spun down at 400 g × 5 minutes and resuspended in ACK red blood cell lysis buffer (Thermo Fisher Scientific, Waltham, MA), then washed for downstream analysis. Cells were stained for NHP-CD45 (clone D058-1283), and immune subset markers (additional marker and clone information available in Supplemental Materials). Upon necropsy, fresh splenocytes, lymph node tissue, and bone marrow tissue were also isolated, stained with the aforementioned antibodies, and fixed in 4% PFA. Flow cytometric data was acquired using an LSR II FACS machine (BD Biosciences. San Jose, CA). Data analysis was performed using FlowJo software (Treestar, San Carlos, CA). Serum was collected by placing blood in an empty tube, letting clot for 20 minutes, then centrifuging at 1000 g × 10 minutes.

### Mixed Lymphocyte Reactions and Stimulation Assays

Fresh isolated splenocytes (responder cells) from primatized mice were collected labeled with Cell Trace Violet (CTV) (Thermo Fisher Scientific. Watham, MA), then plated for 5 days under various stimulatory conditions. Recombinant human IL-2 (PeproTech, Cranbury, NJ) was added to a subset of MLR wells at a concentration of 100 ng/well.

### Tissue Processing and Cell Purification

Experiments were conducted under approval and oversight of the Animal Care and Use Committee of the University of Wisconsin-Madison School of Medicine and Public Health. Non-human primate (NHP) tissue for mouse humanization experiments were obtained and processed as follows. Liver was processed by macerating the tissue over a 100 µm cell strainer with the blunt end of a syringe (both BD Biosciences, San Jose, CA) in sterile DMEM/F12 media (Thermo Fisher Scientific, Waltham, MA). Individualized cells were collected and purified by density centrifugation with Lymphocyte Separation Medium (Corning, Manassas, VA), and red blood cells lysed with ACK buffer (Thermo Fisher Scientific, Waltham, MA). HSPCs were enriched by using NHP-CD34-APC antibody (clone 563, BD Biosciences, San Jose, CA) and MACS anti-APC beads on the QuadroMACS Separator using LS column (Miltenyi, Bergisch Gladbach, Germany). Cells were cryopreserved in CryoStor CS10 freezing medium (Stem Cell Technologies, Vancouver. BC Canada). Thymus was processed from fetal rhesus macaques by placing necropsied thymus in cold, sterile DMEM/F12 media, then removing adipose and other extraneous tissue in a biological safety cabinet. Tissue was dissected into 1 mm × 1 mm fragments with a scalpel and forceps. Fragments were placed in CryoStor CS10 freezing medium, frozen within 4 hours of surgical excision using a controlled rate freezing box to -80° C., then placed in a liquid nitrogen freezer for long-term storage. Human control umbilical cord blood samples were selected using direct conjugated anti-Human CD34 MACS beads. All human tissue research was conducted with informed consent and the approval of University of Wisconsin-Madison (UW) Health Sciences Institutional Review Board.

### Primatization Surgeries and Secondary Transplantation (Further Detail)

The Animal Care and Use Committee of the UW School of Medicine and Public Health approved all experiments. Primatized mice were generated similarly to previous published reports. Briefly, 6-10 week old male and female NBSGW mice were used as immune-compromised host animals. NOG-EXL mice were irradiated with 55 RAD via an X-RAD 320ix irradiator (Precision X-Ray, North Branford, CT). One day prior to primatization surgery. NHP CD34-enriched cells were thawed and plated in SFEM medium plus 100 ng/ml recombinant human Stem Cell Factor (Stem Cell Technologies, Vancouver, BC Canada), and incubated at 37° C. in 5% CO2 overnight. Live cell numbers were determined via hemocytometer and trypan blue method. On the day of surgery, cells were collected, washed and resuspended in 10 mM HEPES-buffered Hank’s balanced salt solution (Thermo Fisher Scientific. Waltham. MA) for tail vein injection. 1×10⁵-1×10⁶ CD34⁺ cells in a 100 µl volume were IV injected into isofluorane anesthetized mice, coinciding with surgery to implant cryopreserved thymus fragments under the left mouse kidney capsule. Mice also received IV injection of αCD2 antibody (100 µg) at days 0 and 7 postsurgery, as previously described in Brown et al. (2018) to deplete passenger T cells emigrating from the thymus fragment. All mice were treated with Buprenorphine SR for post-operative pain management and their drinking water was supplemented with Baytril antibiotic for 10 days post-surgery.

### Histology

Standard brightfield DAB chromogen immunohistochemistry with rabbit monoclonal Anti-CD3G antibody (clone EPR4517. Abeam, Cambridge. MA) and hematoxylin counterstaining was performed on deparaffinized formalin fixed tissue.

### Luminex Magpix Assay

Serum samples were centrifuged at 14,000 × g for 10 minutes. Samples were pooled into 50 uL volumes according to the table below and diluted 1:2 in Assay Diluent. All subsequent steps were conducted using the Monkey Cytokine Magnetic 29-Plex Panel (Invitrogen, Waltham, MA) according to manufacturer instructions and run on the MAGPIX instrument with xPONENT 4.2 software. Analyte concentration values were reported for any samples exhibiting a minimum bead count of 100 microspheres/well and Net MFI were values used to extrapolate concentration data. Standard curves were generated for each analyte using only standards that exhibited 80-120% recovery, based on manufacturer-provided concentration data.

### Mouse Blood Collection and Cellular Immunoprofiling by Flow Cytometry

Peripheral mouse blood was sampled via retro-orbital eye bleeds into heparin-coated capillary tubes (Thermo Fisher Scientifc, Waltham, MA). Blood samples were collected into Eppendorf tubes containing 150 µl of 2% dextran solution (Sigma Aldrich, Saint Louis, MO) in Dulbecco’s phosphate buffered saline without calcium or magnesium (dPBS-/-) (Corning, Manassas. VA) and 150 µl of 0.5%-Heparin solution (Sigma Aldrich). Blood was settled for 20 minutes, then the translucent upper layer (containing leukocytes) was spun down at 400 g × 5 minutes and resuspended in ACK red blood cell lysis buffer (Thermo Fisher Scientific, Waltham. MA) for 10 minutes. Cells were washed twice in cold FACS Buffer (10 mM HEPES-buffered Hank’s balanced salt solution [Thermo Fisher Scientific, Waltham. MA], 2% fetal bovine serum [Hyclone, Pittsburgh, PA] and spun down at 400 g × 5 minutes prior to downstream flow cytometry analysis. Serum was collected by placing blood in an empty sterile tube, letting clot for 20 minutes, then centrifuging at 1000 g × 10 minutes.

Following blood collection. PBMCs were analyzed by flow cytometry for T cell, B cell, and monocyte subsets and activation. Cells were stained for NHP-CD45 (clone D058-1283), CD45RA (clone 5H9), CD3 (clone SP34-2). CD8 (clone RPA-T8), CCR7 (clone 150503), CD4 (clone L200), CD28 (clone CD28.2). CD20 (clone 2H7), CD14 (clone M5E2). IgD (clone L200) (BD Biosciences, San Jose, CA), CD95 (clone DX2), CD27 (clone 0323), CD16 (clone 3G8), HLA-DR (clone L243), CD90 (clone 5E10) (Biolegend. San Diego, CA). CD38 (clone OKT10. Caprico Biotechnologies, Norcross, GA), and Mouse-CD45 (clone 30-F11). Upon necropsy, fresh splenocytes and lymph node tissue were also isolated and stained with the aforementioned antibodies. Unprocessed bone marrow collected from long bones at time of necropsy was stained for NHP CD34 (clone 56), CD38, CD45RA, CD20. CD14, CD90, CD3, and Mouse-CD45 (clones listed above) (Biolegend, San Diego. CA). Additional antibody staining used NHP CD159 (clone Z199; Beckman Coulter, Indianapolis IN), CD117 (clone M-T701, BD Biosciences, San Jose CA), CD66abce (clone TET2, Miltenyi Biotec; Auburn, CA). Flow cytometric data was acquired using an LSR II FACS machine (BD Biosciences, San Jose, CA). Data analysis was performed using FlowJo software (Treestar, San Carlos. CA).

### Mixed Lymphocyte Reactions and Stimulation Assays

Fresh isolated splenocytes from primatized mice were collected and labeled with Cell Trace Violet (CTV) (Thermo Fisher Scientific, Watham. MA) at a concentration of 1 µL CTV per 1×10⁶ cells. These responder cells were then plated at 200,000 cells per well in RPMI + 10% FBS growth media for 5 days under various stimulatory conditions. Allogenic MLR was set up utilizing irradiated, Cell Trace Far Red (CTFR) (Thermo Fisher Scientific. Watham, MA) labeled adult NHP PBMC at a ratio of 2:1 responders to stimulators. Recombinant human IL-2 (PeproTech, Cranbury, NJ) was added to a subset of MLR wells at a concentration of 100 ng/well. Non-MLR stimulatory assays were similarly performed with 200,000 CTV-labeled stimulator cells per well. Phorbol 12-myristate-13-acetate (PMA; 810 nM) with ionomycin (13 µM) (Biolegend, San Diego, CA) or phytohaemagglutinin-L (PHA; 10 µg/mL) (Thermo Fisher Scientific, Watham, MA) were added to each well prior to a 5-day incubation period. Cells were then re-stimulated with PMA (81 nM) and ionomycin (1.3 µM) with Brefeldin A (5 µg/mL) (Biolegend. San Diego, CA) for 5 hours. Following re-stimulation cells were stained for NHP CD45. CD90. CD3. CD8. and CD4 (clones listed above). Intracellular staining was performed for TNF-α (clone MAb11, BD Biosciences, San Jose, CA) and IFN-y (clone B27, Biolegend, San Diego, CA). Cytometric data was similarly acquired with the LSR II cytometer and analyzed with FlowJo software (Treestar, San Carlos, CA).

### Results and Discussion

### Low-Level Engraftment of Adult HSPCs in Transgenic Immune-Deficient Mice

Leveraging humanized mice as a guide, we first sought to engraft a commonly available NHP HSPC source, adult mobilized blood (AMb), in an immune-deficient NSG-variant mouse, the NBSGW (Brown et al., 2018; McIntosh et al., 2015). CD34⁺ cells were sorted from AMb via magnetic beads and IV injected them into naïve NBSGW mice. (FIG. 17A) Peripheral blood engraftment was monitored with flow cytometry for the presence of NHP-CD45+ cells. After 18 weeks, no engraftment of primate cells after infusion of AMb product within this host was detected (data not shown).

NOG-EXL incorporates two transgenic human cytokines (GM-CSF and IL3), both of which have a high degree of homology and subsequent cross-reactivity with NHPs. Given that the NOG-EXL and similar models (e.g., NSG-SGM3, MISTRG) have been shown to enhance human engraftment and chimerism, it was hypothesized that the human-NHP cross-reactive cytokines would be sufficient to enhance the engraftment potential of AMb HSPCs, which is supported by prior publications utilizing adult bone marrow-derived HSPCs within a transgenic murine host (Radtke et al., 2019). IV injection of 1×10⁵-1×10⁶ CD34⁺ AMb cells resulted in moderately durable engraftment in irradiated NOG-EXL mice, though at significantly lower levels of chimerism compared to humanized mice made with umbilical cord-derived CD34⁺ HSPCs (FIGS. 16B,C). Engrafted NHP leukocytes demonstrated CD20⁺ B cell predominance with an absence of T cell chimerism (FIG. 16B, second plot from left. FIG. 16C, middle plot). These results were reproducible (FIG. 16D), indicating the possibility of stable engraftment of adult HSPC populations. However, low-level engraftment and lack of T cell development limits viability of this model iteration for downstream hematopoietic studies or leukocyte functional assays. The paucity of T cells was attributed to the absence of thymic implantation, which is consistent with several reported studies, as well as our own experience injecting CD34+ cord-derived HSPCs alone into adult animals for humanization studies (Brown et al., 2018). Despite the limited durability of this first engraftment attempt, a framework for subsequent experimentation was established. Specifically, an effective transgenic murine host was identified, underscoring the importance of synchronous thymic implantation for the development of a functional peripheral immune compartment.

### Hematopoietic Phenotypes of Adult VS Fetal HSPCs

To enhance the applicability of the model, a more robust and representative primatized immune system was established within the NOG-EXL host. Several sources of HSPCs, including adult bone marrow and fetal liver-derived cells (100 days gestation) in addition to the AMb described above were employed. CD34⁺ cells were magnetically separated and enriched to >90% purity prior to quantification and cytometric phenotyping. These fetal-derived samples revealed a significant enhancement of the CD34(hi) subpopulation compared to AMb, which is known to be associated with augmented engraftment (FIG. 17) (Wu et al., 1999; DiGusto et al., 1996). Additional comparative subset analysis on three samples revealed a potential difference in the percentage of lineage^{neg}CD34^{hi}CD45^{mid}CD117⁺CD45RA⁻ CD90⁺ (group VII) cells, previously described as being the HSPC subset with the greatest hematopoietic potential in NHPs (FIG. 23) (Radtke et al., 2017). Based on these findings, fetal-derived HSPCs were used as the HSPC source for the induction of NHP chimerism in the transgenic host due to their significant CD34(hi) and group VII percentages. Importantly, at this developmental age there was sufficient quantity of CD34+ cells, as well as a developed thymus, to allow for BLT/NeoThy-type primatization of multiple hosts from a single donor.

### Robust Primatization of Transgenic Immune-Deficient Mice

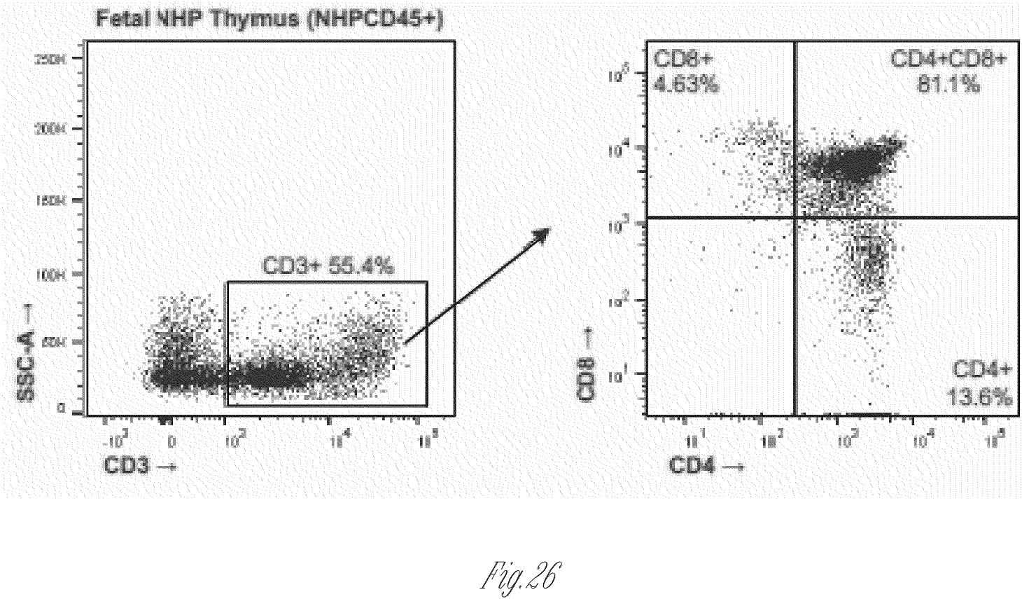

Having identified a promising HSPC source, w 1×10⁶ cryopreserved fetal liver-derived CD34⁺cells were IV injected into the irradiated NOG-EXL host. Additionally, one to two 1 mm × 1 mm of cryopreserved fetal thymus fragments were surgically implanted under the left kidney capsule after cell infusion. To facilitate removal of “passenger thymocytes” known to contribute to graft-vs-host-disease, the recipient mice were injected with human anti-CD2 antibody, which has cross-reactivity in the fetal NHP (FIG. 24). Implementation of this protocol over two separate experiments yielded robust multilineage chimerism of NHP-CD45⁺ cells in all animals (n=6) (FIG. 18A). Engraftment was demonstrated in both CD20⁺ and CD3⁺ lymphocytes, with a significant increase in T cell percentages over time, indicating de novo T cell development via positive and negative selection in the thymic organoid (FIG. 18B). Typical ratios of CD4⁺ and CD8⁺ cells were present in the T cell compartment, with a majority of the cells demonstrating a naive phenotype (CD28⁺CD95⁻), similar to the fetal NHP immune system. This is in contrast to the antigen-experienced NHP adult, which has higher relative percentages of central (CD28⁺CD95⁺) and effector memory (CD28⁻CD95⁺) T cells (FIG. 18C). An interesting observation was made with regard to CCR7 expression on CD4+ T cells in primatized mice and primary NHP fetuses, which demonstrated marked enhanced compared to adult NHPs. CCR7 is known to be involved in thymic homing during murine immune development, raising the possibility that this upregulation could be related to tissue-specific chemotaxis and T cell maturation, however further exploration is warranted (FIG. 25) (Calderon et al., 2011). Also consistent with the fetal immunoprofile, the B cell compartment within our NHP mice was largely naive (CD27⁻IgD⁺), which was similarly less-mature than adult NHP controls (FIG. 18D). NHP-derived CD16^{neg} NK cells, (FIG. 25A), monocytes (FIG. 18E), and granulocytes (FIG. 19F) were also repopulated following engraftment. Furthermore, these animals developed comparable levels of the inflammatory cytokine, IL-12, as well as the chemokines, RANTES and MDC, to adult NHP controls (FIG. 18G). Taken together, these findings demonstrate the robust reconstitution of a diverse, multilineage immune system following hematopoietic engraftment. In contrast to prior murine primatization models, the multilineage nature of this engraftment has wide-reaching implications for the fields of stem cell physiology and immunologic maturation, especially within the context of fetal development and immune function.

### Fetal Thymic Organoid, Secondary Lymphoid Repopulation, and Tissue Resident Immune Cells

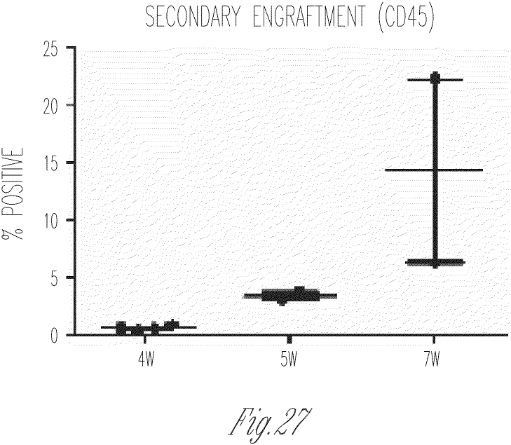

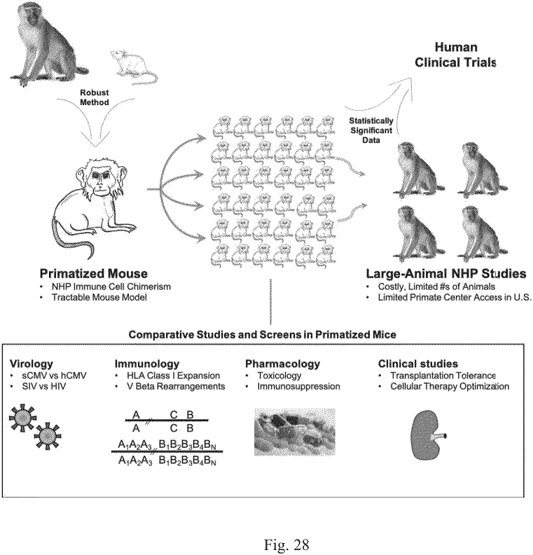

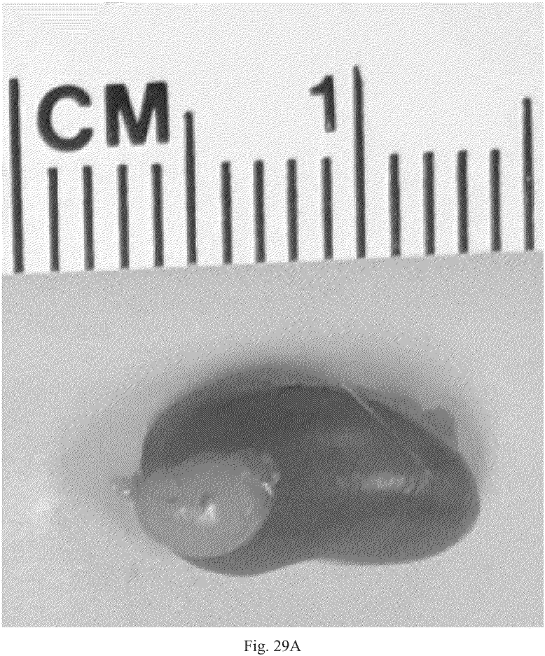

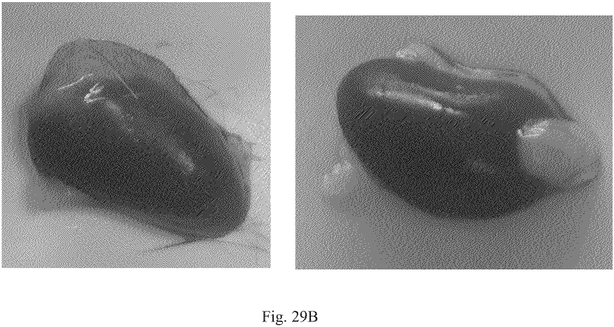

The transplanted fetal NHP thymus fragment produced a large thymic organoid within the kidney capsule (FIG. 19A). The histologic structure was similar to that seen in primary fetal thymus (FIG. 22, middle panel/right), with well-developed Hassall’s corpuscles (FIG. 19C) as well as CD3⁺ thymocytes within the cortical and medullary regions (FIG. 19B). Flow cytometric analysis of processed thymic organoid revealed developing double positive CD4⁺CD8⁺ thymocytes, as well as single-positive CD4⁺ and CD8⁺ T cells (FIG. 19D), which is similar to the immunoprofile of primary fetal thymus (FIG. 26). Importantly, secondary lymphoid tissues (lymph nodes and spleen) were also repopulated with NHP-derived lymphocytes as demonstrated by the presence of CD20⁺ B cells, as well as CD4⁺ and CD8⁺ T cells (FIG. 26B). Furthermore, NHP lymphocytes were found to be tissue resident within end organs involved in immune surveillance and defense, as demonstrated by histologic cross-sections showing lymphocyte aggregation and positive CD3 staining within the intestinal villi (FIG. 26E). Reconstitution of primary and secondary lymphoid organs are a component of immunologic maturation and function, which underscores the utility of this model for future immunologic studies involving acquired immunodeficiencies (e.g. simian immunodeficiency virus) and other lineage specific and non-specific pathologies (e.g. Epstein-Barr virus, cytomegalovirus). Furthermore, the presence of tissue resident lymphocytes opens this model to the study of diverse organ-specific disease processes involving immunologic defense and infectious pathophysiology.

### Bone Marrow Engraftment and Secondary Transplantation

To assess the efficiency of true hematopoietic stem cell (HSC) engraftment within the mouse bone marrow, primatized mice were sacrificed, bone marrow isolated from the femur, and flow cytometric analysis of specific markers associated with HSC identity performed. As expected, the bone marrow contained a rich population of NHP HSPCs, including lineage-negative CD34⁺CD38^{lo}CD45RA^{neg}CD90⁺ cells (FIG. 20A). To test the viability and functionality of these HSPCs, total bone marrow was secondarily transplanted into two strains of transgenic immune deficient mice--NOG-EXLs (FIG. 26) and NSG-SGM3s (FIG. 26B). The NSG-SGM3 variant includes transgenic human SCF in addition to IL-3 and GM-CSF present in the NOG-EXL strain. Importantly, secondary engraftment was evident in both mouse strains, demonstrating the strong engraftment potential of the transplanted HSPC subsets. Interestingly, the addition of human SCF in the NSG-SGM3 model yielded a marked increase in secondary NHP-CD45⁺ chimerism, warranting future primatization studies. The utility of durable primary and secondary engraftment within these models is far-reaching and allows for future studies aimed at evaluating the hematopoietic potential of HSPC subsets and creates the potential for testing novel non-myeloablative conditioning regimens prior to large animal implementation.

### Development of Functional Immune Cells

Having established the durability of engraftment and immune reconstitution, we sought to validate these animals as a translatable model for functional NHP immunologic studies in a series of stimulation assays to characterize the functional fitness of the peripheral T cell compartment. Splenocytes were exposed in vitro to NHP mitogens (PMA/Ionomycin and PHA) to evaluate proliferative capacity of engrafted T cells. After 5 days in culture, stimulated T cells were found to have undergone robust, multigenerational proliferation, thus supporting their ability to respond to non-specific stimuli. Based on these findings, mixed lymphocyte reactions (MLRs) were set up with isolated splenocytes in an attempt to induce an MHC-restricted allo-specific immune response. Indeed, robust T cell activation and proliferation were induced in the presence of MHC-disparate irradiated adult NHP cells. This allogeneic response was further increased upon introduction of the inflammatory cytokine, IL-2, which mimics the hostile post-allogenic transplant environment. Importantly, activated T cells produced IFNγ and TNFα in response to allogeneic stimulation, further demonstrating the development of functional T cells in the NHP mice. These findings have important implications in the field of transplant immunology, which is highly dependent on NHP studies given the disparate nature of small animal and human allo-immune responses. Moreover, the prohibitive morbidity and mortality of inadequately studied human trials is a barrier to the development of effective immunomodulating therapies in transplantation, further emphasizing the importance of effective large animal models. The uncovering of conserved allo-immune responses in these primatized mice, opens the door for extensive experimentation on a proven translational model, thus informing effective large animal studies and subsequent human implementation.

To conclude, a durable primatized mouse model was established by transplanting fetal liver derived CD34⁺ HSPCs and thymic fragments into a cutting-edge and commercially-available transgenic murine host. Although there have been previous reports of mouse primatization, this represents the first model with robust multilineage chimerism and immune reconstitution within multiple physiologic compartments, mirroring that of the donor primate. This model is well-poised for multiple applications within the fields of stem cell physiology, immunologic development, disease pathology, and transplant immunology, all of which currently rely on NHP models. The primatized mouse can be leveraged as an efficient, cost-effective mechanism to inform implementation of novel therapeutic or diagnostic interventions in NHP studies, in transplantation and other contexts (e.g.. virology), thus minimizing the risk and logistical burden of large animal models. Further, creation of primatized mice with other NHP species (e.g., cynomologus macaques), and/or utilization of neonatal tissues for primatization of NeoThy animals will enable novel analyses of species-specific immune responses and ontogeny-associated immune cell development and function, respectively. By accelerating the pace of NHP studies through the utilization of this primatized model, we hope that impactful and potentially life-saving approaches can be more rapidly translated to the bedside, thus efficiently advancing the field of clinical medicine.

### EXAMPLE 5

Rhesus macaque non-human primatized (NHP) mice were achieved with low numbers of input CD34+ cells. Rhesus macaques were used to create NHP mice via injection of 114,000 CD34+ cells from fetal liver with surgical transplantation of fetal thymus fragment (1 fragment). Primatized mice peripheral blood was sampled at 12 weeks post-primatization, stained for overall NHP chimerism (CD45), NHP B cells (CD20) and NHP T cells (CD3). NSG-SGM3 immune deficient mouse strains were used, with successfully NHP chimerism despite the low cell # injected at day0.

TABLE 1

12 weeks

| | Mouse | Adjusted NHPCD45 | NHPCD20 (%) | NHPCD3 (%) |
|---|---|---|---|---|
| NSG-SGM3 | 169228N | 77.3 | 1.1 | 57.1 |
| NSG-SGM3 | 169229L | 58.7 | 6.1 | 23.3 |
| NSG-SGM3 | 169229RR | 21.8 | 1.9 | 34.9 |

Cynomologus macaque primatized (NHP) mice were prepared. Cynomologus macaques were used to create NHP mice via injection of 330,000 CD34+ cells from fetal liver with or without surgical transplantation of fetal thymus fragment (1 fragment). Primatized mice peripheral blood was sampled at 14-16 weeks post-primatization, stained for overall NHP chimerism (CD45), NHP B cells (CD20) and NHP T cells (CD3) . Multiple immune deficient mouse strains were used, including the NBSGW which does not have exogenous cytokines, and all were successfully primatized robustly. Importantly, when no thymus was transplanted (i.e., cells only) in mouse 1200N there was limited T cell engraftment, indicating the importance of the thymic organoids for robust T cell development.

TABLE 2

| 14weeks | | | | | | | | 16 weeks | | | | | |
|---|---|---|---|---|---|---|---|---|---|---|---|---|---|
| | | Mouse | hCD45 | mCD45 | Adjusted NHPCD45 | nhpCD20 | nhnCD3 | Mouse | hCD45 | mCD45 | Adjusted NHPCD45 | nhpCD20 | nhnCD3 |
| | NOG-EXL | 1191LLR | 74.0% | 23.0% | 76.3 | 40.0% | 55.0% | 1194N | 62.4% | 27.7% | 69.3 | 70.3% | 26.3% |
| | NBSGW | 1194N | 71.1% | 25.4% | 73.7 | 86.1% | 6.9% | 1200N | 84.1% | 11.2% | 88.2 | 87.8% | 1.7% |
| | NBSGW | 1194L | 94.4% | 2.4% | 97.5 | 27.7% | 60.4% | - | - | - | - | - | - |
| no thymus | NBSGW | 1200N | 79.5% | 18.7% | 81.0 | 91.5% | 1.0% | - | - | - | - | - | - |

### REFERENCES

Baenziger et al., Proc. Natl. Acad. Sci. USA. 103:15951 (2006).
Binhaiem et al., Lab Investig., 75:339 (1996).
Brown et al., Stem Cell Reports, 10:1175 (2018).
Burlingham et al., N. Engl. J. Med., 339:1657 (1998).
Calderon et al., Proc. Natl. Acad. Sci. USA. 108:7517 (2011).
De Jong et al., Blood, 86:4076 (1995).
DiGiusto et al., Blood, 87:1261 (1996).
Donahue et al., Nature, 321:872 (1986).
Donahue et al., Science. 241:1820 (1988).
Evans & Silvestri. Curr. Opin. HIV AIDS, 8:255 (2013).
Herndler-Brandstetter et al., Proc. Natl. Acad. Sci. USA. 114:E9626 (2017).
Holman et al., PLoS One. 12:e0179039 (2017).
Honeycutt et al., J. Clin. Invest., 128:2862 (2018).
Huang et al., Xenotransplantation, 24:______ (2017).
Kalscheuer et al., Sci. Transl. Med., 4:125ra30 (2012).
Kamel-Reid et al., Science, 242:1706 (1988).
Lan et al., Blood, 108:487 (2006).
Larochelle et al., Blood, (117):1550 (2011).
Markus. Trends Parasitol., 32:271 (2016).
Maufort et al., Nat. Comm., ______:______ (2018).
Mayer, et al., Blood, 74:613 (1989).
McIntosh & Brown, Chimerism, 3:6:40 (2015).
McIntosh et al., Stem Cell Reports, 4:171 (2015).
Melkus et al., Nat. Med., 12:1316 (2006).
Mold et al., Science, 330:1695 (2010).
Mosier et al., Nature, (335):256 (1988).
Nadon, J. Gerontol., 61:813 (2006).
Nguyen et al., PLoS Pathog., 13:el006378 (2017).
Pearson et al., Curr. Protoc. Immunol., Chapter 15, Unit-15.21 (2008).
Prescot et al., Drug Disc., 23:51 (2017).
Radtke et al., Exp. Hematol., 70:31 (2019).
Radtke et al.. Science Translational Medicine., 9, eaan1145. (2017).
Rongvaux et al., Nat Biotechnol. 32:364 (2014).
Rosenzweig et al., J. Med. Primatol., 30:36 (2001).
Simon et al., Proc. Biol. Sci., 282:20143085 (2015).
van Gils et al., Exp. Hematol., 22:248 (1994).
Wiseman et al., ILAR J., 54:196 (2013).
Wiseman et al., J. Virol., 81:349 (2006).
Wiseman et al., Nat. Med., 15:1322 (2009).
Wognum et al., Blood. 86:581 (1995).
Wognum et al., Leuk. Lymphoma. 24:11 (1996).
Wong et al., Science, 228:810 (1985).
Wu et al., Pediatr. Res., 46:163 (1999).
Yang et al., Cell, 47:3 (1986).
Zanjani et al., Blood, 79:3045 (1992).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method of making a primatized rodent or swine, comprising:
providing an immune deficient rodent or swine lacking mature T cells, B cells and/or NK cells, which rodent or swine optionally expresses primate IL3 and/or primate GM-CSF;
providing a population of cells from a fetal liver of a non-human primate which population comprises isolated CD34+ cells or which population is depleted of CD3+ cells, or a population which comprises hematopoietic stem or progenitor cells obtained from induced pluripotent stem cells or embryonic stem cells;
providing at least one portion of a thymus from a fetal, neonatal or adult non-human primate; and
introducing an amount of the population of cells and the at least one portion of the
thymus into the rodent or swine so as to provide a primatized rodent or swine.

2. The method of claim 1 wherein the primatized rodent or swine comprises mature T cells, B cells and/or NK cells.

3. The method of claim 1 wherein the immune deficient rodent or swine lacks mature T cells, B cells and NK cells.

4. (canceled)

5. The method of claim 1 wherein the non-human primate is a monkey.

6-7. (canceled)

8. The method of claim 1 wherein the thymus is introduced to a kidney or an ear of the rodent or swine.

9. The method of claim 1 wherein the source of the cells and the thymus is the same.

10-11. (canceled)

12. The method of claim 1 wherein the rodent or swine is immune deficient as a result of irradiation or one or more genetic mutations.

13. (canceled)

14. The method of claim 1 wherein the rodent or swine expresses human IL3 and/or human GM-CSF and/or human SCF.

15-16. (canceled)

17. The method of claim 1 wherein the at least one portion of the thymus is about 0.05 mm × 3 mm, about 0.5 mm × 2 mm or about 1 mm × 1 mm or wherein the population comprises about 0.05 × 10⁵ to about 9 × 10⁵ cells, about 0.1 × 10⁵ to about 7.5 × 10⁵ cells or about 0.5 × 10⁵ to about 1.5 × 10⁵ cells.

18-19. (canceled)

20. The method of claim 1 wherein the CD34+ cells are enriched using beads or density centrifugation.

21. (canceled)

22. The method of claim 1 wherein the rodent is a NCG, NSG or NOG mouse.

23. The method of claim 1 wherein the rodent is a NSG-SGM3, NOG-EXL NBSGW, NSG-IL15. or NOG-1L6 mouse.

24. (canceled)

25. The method of claim 1 wherein the engraftment efficiency is at least about 20% or up to 65%.

26. (canceled)

27. The method of claim 1 wherein the thymus is from a fetus and the population comprises isolated CD34+ cells or is depleted of CD3+ cells.

28. The method of claim 1 wherein the population comprises the hematopoietic stem or progenitor cells.

29. A primatized rodent or swine comprising non-human primate T cells, B cells and/or NK cells and a portion of a thymus from a fetal non-human primate, whicb rodent or swine optionally expresses primate IL3 and/or primate GM-CSF.

30-31. (canceled)

32. The rodent or swine of claim 29 which is a cynomolgus macaque or a rhesus macaque.

33. The rodent or swine of claim 29 wherein the thymus is proximal to the kidney.

34. (canceled)

35. The rodent or swine of claim 29 wherein the cells and the thymus are allogeneic or xenogenic.

36. (canceled)

37. The rodent or swine of claim 29 wherein the rodent or swine expresses human IL3 and/or human GM-CSF and/or human SCF.

38-39. (canceled)
